(19) United States
(12) Patent Application Publication
(10) Pub. No.: US 2023/0172504 A1
(43) Pub. Date: Jun. 8, 2023

(54) BLOOD COLLECTION DEVICES, SYSTEMS, AND METHODS

(71) Applicant: Avia Vascular, LLC, Salt Lake City, UT (US)

(72) Inventors: Kevin Jerry Cook, Kaysville, UT (US); Jay Allen Muse, Salt Lake City, UT (US)

(73) Assignee: Avia Vascular, LLC, Salt Lake City, UT (US)

(21) Appl. No.: 18/106,930

(22) Filed: Feb. 7, 2023

Related U.S. Application Data
(63) Continuation of application No. PCT/US2021/045290, filed on Sep. 8, 2021.
(60) Provisional application No. 63/062,827, filed on Aug. 7, 2020.
(60) Provisional application No. 63/068,610, filed on Aug. 21, 2020.

(51) Int. Cl. A61B 5/15; A61B 5/153

(52) U.S. Cl. CPC A61B 5/150992; A61B 5/153

(57) ABSTRACT

A blood collection system can include a cannula for insertion into a catheter system that comprises a hub with an internal chamber, a distal port in fluid communication with the internal chamber and coupled with a catheter tube that can be preplaced in a blood vessel of a patient, and an access port in fluid communication with the internal chamber. The cannula can be inserted in the catheter tube and can include a distal opening that is positioned within the blood vessel of a patient and is in fluid communication with an interior of the blood vessel to permit blood to flow into a lumen of the cannula, and can include a proximal opening positioned within the internal chamber to permit blood to flow from the cannula into the internal chamber and through the access port of the hub.

### CROSS-REFERENCE TO RELATED APPLICATIONS

This application is a continuation of Patent Cooperation Treaty (PCT) International Application No. PCT/US2021/045290, filed Aug. 9, 2021, titled BLOOD COLLECTION DEVICES, SYSTEMS, AND METHODS, which claims priority to U.S. Provisional Patent Application No. 63/062,827, filed Aug. 7, 2020, titled BLOOD COLLECTION DEVICES, SYSTEMS, AND METHODS, and U.S. Provisional Patent Application No. 63/068,610, filed Aug. 21, 2020, titled BLOOD COLLECTION DEVICES, SYSTEMS, AND METHODS, the entire contents of each of which are hereby incorporated by reference herein.

### TECHNICAL FIELD

Certain embodiments described herein relate generally to devices, systems, and methods for blood collection and further embodiments relate more particularly to devices, systems, and methods for facilitating blood collection via a previously placed catheter, such as, for example, a peripheral intravenous catheter.

### BACKGROUND

Known devices, systems, and methods for drawing blood, including drawing blood using a previously placed catheter, suffer from a variety of drawbacks. Embodiments disclosed herein remedy, ameliorate, or avoid one or more of such drawbacks. Other or further uses and methods are also disclosed.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1A is a schematic cross-sectional view of a proximal portion of an embodiment of a base catheter system placed within a patient;

FIG. 1B is a schematic cross-sectional view of a distal end of the base catheter system of FIG. 1A within a blood vessel of a patient;

FIG. 2A is a cross-sectional view of an embodiment of an access system coupled with an embodiment of a base catheter system;

FIG. 2B is an enlarged cross-sectional view of a distal portion of the coupled access system and base catheter system of FIG. 2A taken along the view line 2B in FIG. 2A;

FIG. 3 is a perspective view of the access system of FIG. 2A;

FIG. 4 is a perspective view of an embodiment of a cannula assembly that can be included in embodiments of the access system of FIG. 2A;

FIG. 5 is a perspective view of an embodiment of an obturator assembly that can be included in embodiments of the access system of FIG. 2A;

FIG. 6 is a perspective view of another embodiment of an access system;

FIG. 7 is a cross-sectional view of a proximal portion of the access system of FIG. 6, the cross-sectional view being taken along a plane parallel to the plane of the page of FIG. 6 and extending through a longitudinal axis of the access system;

FIG. 8 is a perspective view of an embodiment of a cannula assembly that can be included in embodiments of the access system of FIG. 6;

FIG. 9 is an enlarged perspective view of a distal end of the cannula assembly of FIG. 8;

FIG. 10 is a perspective view of an embodiment of a valve assembly that can be included in embodiments of the access system of FIG. 6;

FIG. 11A is a cross-sectional view of a portion of the access system taken along the plane described with respect to FIG. 7, with the valve assembly depicted in a closed configuration;

FIG. 11B is another cross-sectional view of the portion of the access system taken along the plane described with respect to FIG. 7, with the valve assembly depicted in an open configuration;

FIG. 12 is a perspective view of another embodiment of an access system that includes a guide member;

FIG. 13 is a cross-sectional view of the guide member;

FIG. 14 is a perspective view of another embodiment of an access system;

FIG. 15A is a cross-sectional view of a proximal portion of the access system of FIG. 14, the cross-sectional view being taken along a plane parallel to the plane of the page of FIG. 14 and extending through a longitudinal axis of the access system, the access system including an embodiment of a valve assembly that is depicted in a closed configuration;

FIG. 15B is another cross-sectional view of the proximal portion of the access system taken along the plane described with respect to FIG. 15A, with the valve assembly depicted in an open configuration;

FIG. 16 is a perspective view of an embodiment of a closed catheter system;

FIG. 17 is a perspective view of the closed catheter system of FIG. 16 with a needle portion being separate from a hub and catheter portion thereof;

FIG. 18 is a cross-sectional view of a portion of the closed catheter system of FIG. 16 in an operative state in which the needle has been removed from hub and catheter;

FIG. 19 is a cross-sectional view of an embodiment of an access assembly in an assembled stated and positioned within the portion of the closed catheter system depicted in FIG. 18;

FIG. 20A is a cross-sectional view of another embodiment of an access system coupled with an embodiment of a base catheter system;

FIG. 20B is an enlarged cross-sectional view of a distal portion of the coupled access system and base catheter system of FIG. 20A taken along the view line 20B in FIG. 20A;

FIG. 20C is a proximally directed end-on elevation view of the distal portion of the coupled access system and base catheter system of FIG. 20A;

FIG. 21 is an enlarged cross-sectional view of a distal portion of another embodiment of an access system coupled with an embodiment of a base catheter system similar to the view depicted in FIG. 20B;

FIG. 22 is a perspective view of another embodiment of a base catheter system and another embodiment of an access system shown in a disassembled state;

FIG. 23 is a perspective view of a portion of an embodiment of an extension set that can be included in embodiments of an access system, such as the access system of FIG. 22;

FIG. 24 is a perspective view of an embodiment of an obturator that can be included in embodiments of an access system, such as the access system of FIG. 22;

FIG. 25 is a perspective view of an embodiment of a cannula that can be included in embodiments of an access system, such as the access system of FIG. 22;

FIG. 26A depicts a stage of an illustrative method of using an embodiment of an access system, wherein a perspective view of an embodiment of a base catheter system coupled with an embodiment of an extension set is shown;

FIG. 26B depicts another stage of an illustrative method of using an embodiment of an access system, wherein a perspective view of an embodiment of an access assembly inserted through a portion of the extension set and into the base catheter system is shown, the access assembly including an embodiment of a cannula and further including an embodiment of an obturator positioned within the cannula;

FIG. 26C depicts another stage of an illustrative method of using an embodiment of an access system, wherein a perspective view of an embodiment of an obturator having been removed from an embodiment of a cannula that remains coupled with an extension set that is coupled with a base catheter system is shown;

FIG. 27 is a perspective view of an embodiment of an access assembly in a disassembled state, the access assembly including an embodiment of a cannula and further including an embodiment of an obturator configured to be positioned within the cannula; and

FIG. 28 is a cross-sectional view of the access assembly of FIG. 27 in an assembled stated and positioned within a closed catheter system.

### DETAILED DESCRIPTION

Embodiments of the present disclosure relate generally to devices, systems, and methods for blood collection (also referred to as blood draws, blood aspiration, phlebotomy procedures, etc.). The blood collection can be achieved via an access system that includes a conduit or cannula that is inserted into a previously placed catheter, such as a catheter tube of a previously placed catheter system, such as, for example, a previously placed peripheral intravenous (PIV) catheter. In certain embodiments, the access system can provide a desirable alternative to venipuncture.

In one context, fluid, particularly blood, is drawn from patients on a routine basis in many hospitals, clinics, and laboratories. One of the most common ways to draw blood is venipuncture, which is a method that involves inserting a needle through the skin and into an underlying vein. In some instances, blood can be drawn as frequently as every six hours. Further, patients can be subjected to multiple attempts each time a needle is inserted into the skin, and the more frequent the withdrawals become, the more difficult it can become to find a location for the next withdrawal. Each attempt can be painful and a nuisance. Other options for the withdrawal of blood and other fluids, however, are limited, and can often be even more painful than venipuncture. Some of these options include the use of peripherally inserted central catheters (PICC lines), central lines, repeated peripheral venipuncture, and groin sticks.

In some instances, blood may be drawn via a peripheral intravenous catheter at the time of insertion. In many instances, however, clots or fibrin sheaths can form at the tip of an intravenous catheter over time, so it is generally not desirable to draw blood from peripheral intravenous catheters, as previously designed, at any significant time after placement. In various instances, the obstructions that form at the distal tip can fully obstruct the distal tip, thereby entirely preventing withdrawal of blood through the catheter. In other instances, the obstructions may only partially obstruct the distal tip, but may affect fluid flow through the catheter in such a way as to promote hemolysis and/or otherwise reduce the quality of the blood withdrawn through the catheter and/or reduce a speed of the withdrawal.

In some instances, the intravenous catheter can lack sufficient rigidity to remain fully patent during a blood draw. For example, in some instances, the catheter is formed of a material that softens over time when within the patient vasculature. While blood draws may be possible upon initial placement of the catheter, blood draws may become increasingly difficult and ultimately impossible after the initial placement as the catheter softens. The catheter lumen may collapse when negative pressure applied at a proximal end of the catheter for an attempted blood draw, thereby inhibiting or preventing blood withdrawal.

In certain instances, even when blood is able to be withdrawn through a softened catheter, the quality of the drawn blood, relative to the quality of blood drawn immediately after placement of the catheter, may deteriorate as the catheter softens. Without being bound by theory, this quality reduction may be due to the lumen defined by the catheter becoming more tortuous or otherwise changing shape as the catheter softens. For example, upon initial placement, the catheter may have relatively few curves and/or regions of the catheter that extend through the skin and the vessel wall may define a rounded cross-sectional profile. Over time, the softening or softened catheter may become compliant so as to conform to tortuous anatomy through which it passes and/or the cross-sectional profile at the insertion regions may flatten or otherwise change shape. Blood drawn through these altered regions may, for example, be more susceptible to hemolysis.

In still other or further instances, an opening at the catheter tip may suction against a feature of the vessel anatomy, such as a valve or the vessel wall, during an attempted blood draw, thereby preventing blood withdrawal through the opening.

For one or more of the foregoing reasons and/or for other reasons, it can be desirable to provide a conduit or cannula through a previously placed catheter (e.g., a PIV catheter) to achieve a high-quality blood draw through the catheter. In some instances, the cannula can have sufficient rigidity to provide a patent lumen through which blood can readily pass. In other or further instances, the cannula can straighten or otherwise reduce a tortuousness of a path along which the drawn blood passes, which may yield a laminar flow of blood through the cannula. In other or further instances, the distal end of the cannula may be placed distally relative to the tip of the previously placed catheter, which may, for example, avoid interference from clots or fibrin sheaths; permit access to a region beyond an otherwise obstructing anatomical feature, such as by moving past one or more venous valves and/or away from a vessel wall; permit movement to a region of increased blood flow; and/or move away from a region of vascular trauma due to venipuncture and catheter insertion, any or all of which can result in substantially improved blood draws, such as, for example, by establishing blood flow and/or by reducing hydrolysis or hemolysis of the sampled blood.

Certain embodiments disclosed herein can remedy, ameliorate, or avoid one or more limitations or drawbacks of known systems in which a catheter is inserted through a previously placed catheter for purposes of blood collection. One or more of these and/or other advantages will be apparent from the present disclosure.

In some instances, a catheter is introduced into a patient, and subsequently, a cannula is introduced into the patient through the catheter. The catheter may be referred to herein as a placed catheter, a preplaced catheter, an anchor catheter, or a base catheter. The cannula may, in some instances, also be referred to as a conduit, a fluid channeler, a fluid extraction member, etc. In many instances, the cannula will have properties different from the placed catheter. For example, in some instances, at least a portion of the cannula may be stiffer or more rigid than the placed catheter. The cannula may, in some instances, define an open lumen or passageway through which blood can pass from the vessel and out of the patient through the cannula into any suitable blood collection device. The cannula may, in some instances, straighten or otherwise reduce a tortuous path of a lumen defined by the catheter. In some instances, the cannula may be advanced past a distal end of the placed catheter.

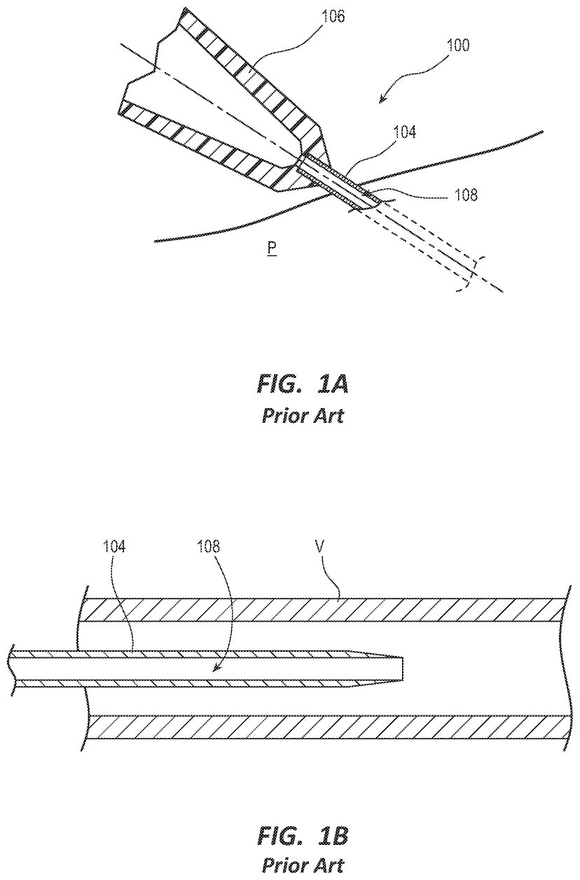

FIGS. 1A and 1B depict an embodiment of a base catheter system 100, such as, for example, a peripheral intravenous (PIV) catheter system. The base catheter system 100 may also be referred to herein as a placed catheter system, a preplaced catheter system, an anchor catheter system, etc. The base catheter system 100 includes a catheter tube 104 and a hub 106 that is fixedly secured to a proximal end of the catheter tube 104. The base catheter system 100 can be inserted into a patient P in any suitable manner such that at least a distal end of the catheter tube 104 extends into a vessel V (e.g., a vein) of the patient (FIG. 1B) and such that a proximal portion of the base catheter system 100, including the hub 106, is accessible at an exterior of the patient. The catheter tube 104 can define a lumen 108 through which, for example, infusions may be delivered into the vessel V. The hub 106 may be of any suitable variety. In some embodiments, the hub 106 is a female luer connector.

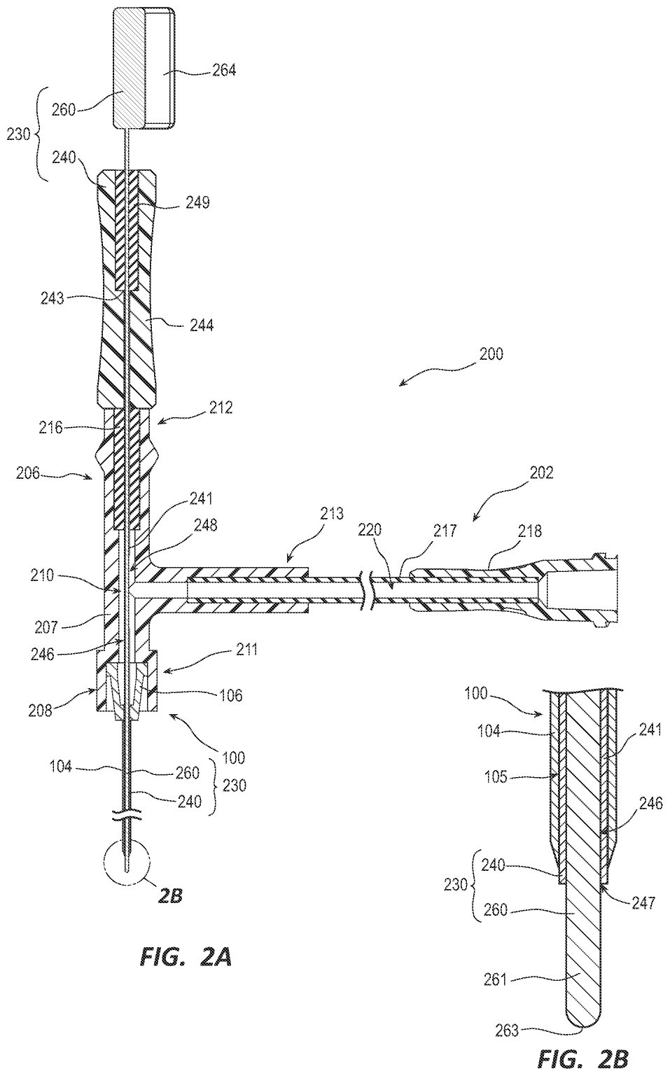

With reference to FIGS. 2A-2C, in some embodiments, an access system 200 can be configured to couple with the base catheter system 100. The access system 200 can also be referred to as a fluid channeling system, a fluid extraction system, a blood removal system, a follow-on cannula system, etc. The base catheter system 100, such as the catheter tube 104 thereof, can be inserted into a vessel of a patient in any suitable manner, such as previously described. One or more portions of the access system 200 may be coupled to the base catheter system 100. In some instances, no portion of the access system 200 is coupled with the base catheter system 100 prior to insertion of the base catheter system 100 into the patient. In other instances, at least a portion of the access system 200 (such as, e.g., an extension set) may be coupled with the base catheter system 100 prior to insertion of the base catheter system 100 into the vasculature of the patient.

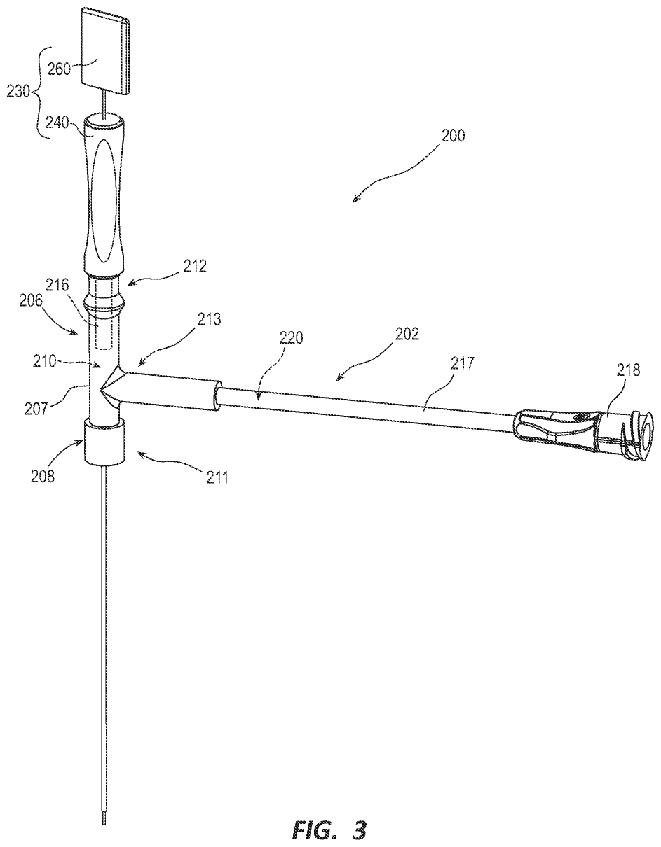

FIGS. 2A and 2B show the illustrated access system 200 fully coupled with the base catheter system 100. For example, FIGS. 2A and 2B can be illustrative of a stage of use of the access system 200 at a time after the base catheter system 100 has been inserted into the vasculature of the patient and after the access system 200 has been coupled with the base catheter system 100, but before fluid is withdrawn from the vessel of the patient via the access system 200. FIG. 3 is a perspective view of the access system 200 in a decoupled state, relative to the base catheter system 100. In some instances, the access system 200 may be coupled to the base catheter system 100 when in the preassembled state, as shown. In other instances, separate components of the access system 200 may be coupled to the base catheter system at various stages of using the access system 200, as further discussed below.

With reference to FIGS. 2A, 2B and 3, in the illustrated embodiment, the access system 200 includes an extension set 202 that can be attached to the hub 106 of the base catheter system 100. The extension set 202 can include a hub 206 that includes any suitable connector 208 configured to interface with the hub 106 of the base catheter system 100 and thereby connect the extension set 202 to the hub 106. For example, in some embodiments, the connector 208 can comprise a luer connector, a luer lock connector, or any other suitable connector, which, in further embodiments, may provide a fluid seal with the hub 106. For example, in some embodiments, the hub 106 is formed as a female luer, and the connector 208 is formed as a male luer or as a locking male luer.

In various embodiments, the hub 206 may include handle or housing 207. In the illustrated embodiment, the housing 207 of the hub 206 defines an open interior, cavity, or internal chamber 210. As discussed hereafter, the chamber 210 may permit fluid flow therethrough, such as to permit blood that has egressed from the vessel to pass therethrough during a blood withdrawal and/or to permit saline to pass therethrough in a substantially opposite direction during flushing. The hub 206 may include a first port 211, a second port 212, and a third port 213, each of which may be in fluid communication with the chamber 210.

In the illustrated embodiment, the first port 211 is positioned at a distal end of the hub 206, and may be referred to as a distal port or as a connection port. The connector 208 may be positioned at or about the first port 211. Blood from the vessel may enter the hub 206 through the first port 211 and/or infused fluid may exit from the hub 206 through the first port 211.

In the illustrated embodiment, the second port 212 is positioned at a proximal end of the hub 206. In various embodiments, the second port 212 may be referred to as a proximal port, or as a valved port. The term “proximal port” does not necessarily imply that the port is the proximal-most port, but rather, that the port is positioned proximally relative to the first port or distal port 211. In the illustrated embodiment, the second port 212 includes a valve, septum, or sealing member 216. As further discussed below, various components of the access system 200 may be introduced through and/or withdrawn from the second port 212. The sealing member 216 can prevent fluid from escaping from the chamber 210 via the second port 212.

The third port 213 may be referred to as an access port. In the illustrated embodiment, the third port 213 is positioned at a lateral position relative to a longitudinal axis of the hub 206. The third port 213 may alternatively be referred to as a side port or lateral port. The third port 213 may permit blood withdrawals, infusions of any suitable variety, and/or saline flushes therethrough. Stated otherwise, blood obtained from the vessel may exit from the hub 206 through the third port 213 and/or infusion fluids may enter the hub 206 through the third port 213.

In the illustrated embodiment, the third port 213 extends substantially orthogonally relative to a longitudinal axis through the first port 211 and the second port 212. Stated otherwise, the hub 206 may include a substantially T-shaped arrangement of ports, and may, in some instances, be referred to as a T-lock extension set. Other arrangements are contemplated. For example, in some instances, the third port 213 may extend at an oblique angle relative to the longitudinal axis through the first and second ports 211, 212. The hub 206 may be substantially Y-shaped, and the extension set 202 may, in some instances, be referred to as a Y-lock extension set.

In some embodiments, an extension tube 217 is connected to the third port 213. Any suitable tubing is contemplated. The extension tube 217 can be attached at a proximal end thereof with any suitable connector 218. The connector 218 can be configured to couple with any suitable fluid source and/or fluid collection device or may, in other embodiments, be integrally formed with fluid source and/or fluid collection apparatus. For example, in the illustrated embodiment, the connector 218 is formed as female luer connector configured to couple with a syringe, which may be used for infusion or blood collection. The connector 218 may also or alternatively be used for connection to blood draw apparatus, such as syringes or evacuated blood collection tubes (e.g., Vacutainer® tubes available from Becton Dickinson) and/or apparatus therefor. For example, in some embodiments, the connector 218 may include or may be configured to connect with a Vacutainer® one-use holder, via which blood draws may be made into one or more Vacutainer® tubes.

In the illustrated embodiment, the connector 218 does not provide any valving relative to a lumen 220 that extends through the extension tube 217. In some instances, a clamp, such as a standard sliding clamp or a standard thumb-actuated compression clamp (not shown) may selectively prevent or permit fluid flow through the extension tube 217. In other embodiments, the connector 218 may comprise a valve and/or a clamp may be omitted. For example, in some embodiments, the connector 218 comprises a needleless connector valve of any suitable variety.

Although the extension set 202 has been identified as a component of the access system 200, in other or further instances, the extension set 202 can be considered as a component of the base catheter system 100. For example, in some embodiments, the base catheter system 100 can be termed an open catheter system 100, such as an open intravenous catheter system. With reference again to FIG. 2A, the hub 106 of the base catheter system 100 can include a female luer connector that is devoid of any sealing member, such that upon initial insertion of the catheter tube 104 into a vessel of a patient, blood may be permitted to egress from the hub 106. In some embodiments, the extension set 202 may be coupled with the hub 106, and may effectively extend a fluid pathway of the base catheter system 100 to the connector 218, which may likewise be open. As previously noted, however, in some embodiments, a clamp or other valving member of any suitable variety may be used to selectively close the extension tube 217. In any event, the extension set 202 may be considered a part of the base catheter system 100, and in some instances, may be considered as a component of and/or as being coupled with a closed catheter system.

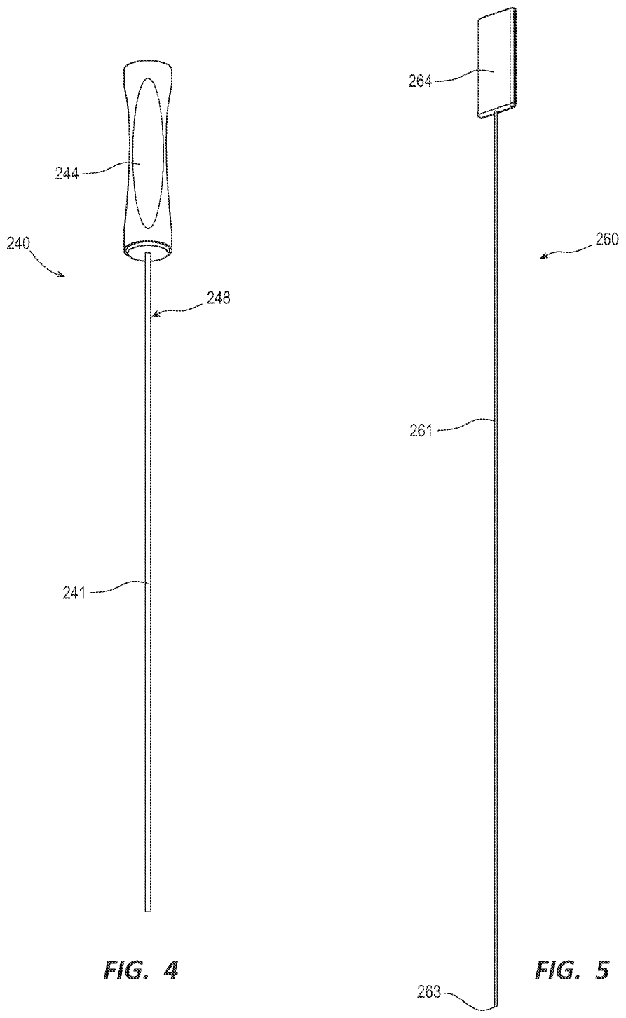

With reference to FIGS. 2A, 2B, 3, 4, and 5, the access system 200 can include an access assembly 230. In the illustrated embodiment, the access assembly 230 includes a cannula assembly 240 that includes a cannula 241, which may also be referred to as a fluid channeler, a fluid extraction member, a thin-walled cannula, etc., and an obturator assembly 260, which includes an elongated obturator 261.

With reference to FIGS. 2A, 2B, 3, and 4, the cannula 241 can comprise a thin-walled member of formed of any suitable material. For example, in various embodiments, the cannula 241 comprises a metallic tube (e.g., stainless steel) or a plastic tube (e.g., extruded plastic). In some embodiments, the cannula 241 is relatively rigid or is less laterally flexible than the catheter tube 104. The cannula 241 can provide structural support to the catheter tube 104 when inserted therein, or stated otherwise, can itself maintain a lumen 246 that passes through the cannula 241 in an open configuration when the cannula 241 is inserted through the catheter tube 104. In some embodiments, the cannula 241 can be sufficiently rigid to maintain a substantially rectilinear orientation when inserted through the catheter tube 104 when placed in the patient such that, even when positioned through the skin and into a vessel of the patient, the cannula 241 is substantially rectilinear. In other embodiments, the cannula 241 may be sufficiently rigid so as to curve only slightly, or may be said to curve or bend substantially less than the catheter tube 104 while extending through the skin and into the vessel of the patient. Stated otherwise, the cannula 241 may straighten, render less curved or angled, unkink, or otherwise reconfigure the catheter tube 104 to be more rectilinear when the cannula 241 is inserted through at least a portion of the lumen 105 of the catheter tube 104. In various instances, the relatively straightened, less curved, and/or unkinked configuration of the cannula 241 when extending through the placed catheter tube 104 can provide a laminar flow of blood through a lumen 246 of the cannula 241, which can provide a better blood sample. For example, blood samples obtained through the cannula 241 may be free or relatively free of hemolysis.

The cannula 241 can include a distal opening or distal port 247 at a distal end of the cannula 241 that is in fluid communication with the lumen 246. In the illustrated embodiment, the distal port 247 is positioned at the distalmost tip of the cannula 241. In other embodiments, the port 247, or one or more additional ports 247, can be positioned at the side of the distal tip of the cannula 241, such as in a lateral orientation and/or proximal to the distal tip.

The cannula 241 can further include a proximal opening or proximal port 248 at a more proximal portion of the cannula 241 that is in fluid communication with the lumen 246. In some embodiments, the proximal port 248 may be positioned at an intermediate region of the cannula 241, and thus the term “proximal port” does not necessarily connote that the port is at a proximal-most end of the cannula 241. Rather, the proximal port 248 is positioned proximally relative to the distal port 247. In some embodiments, the proximal port 248 may alternatively be referred to as an intermediate port.

In the illustrated embodiment, the proximal port 248 is relatively elongated and is positioned at a side of the cannula 241. The proximal port 248 may be said to extend through a sidewall of the cannula 241. The proximal port 248 may also be referred to as a side port. In the illustrated embodiment, the proximal port 248 is positioned such that when the cannula 241 is inserted (e.g., fully inserted) into or through the catheter tube 104, such as in a manner that puts the distal port 247 within the patient and in fluid communication with the interior of the vessel of the patient, the proximal port 248 is positioned external to the patient and within the chamber 210 of the hub 206. Accordingly, blood may enter through the distal port 247, pass through the lumen 246, and egress through the proximal port 248 into the chamber 210 of the hub 206 during blood collection. The blood may then pass through the third or side port 213 of the hub, through the extension tube 217, and through the connector 218 into whatever blood collection device has been coupled with the connector 218. In other embodiments, the cannula 241 can include a plurality of proximal ports 248.

In some embodiments, the access assembly 230 may only be comprised of the cannula assembly 240. For example, the cannula 241 of the cannula assembly 240 may be inserted through the hub 206 and through the placed catheter system 100, e.g., to collect blood. The cannula 241 may subsequently be removed from the hub 206. In other embodiments, the obturator assembly 260 may be used with the cannula assembly 240, in manners such as described hereafter. The obturator assembly 260 can, in some embodiments, inhibit or prevent the distal tip of the cannula 241 from scraping, piercing, tearing, or otherwise damaging the catheter tube 104 during insertion of the access assembly 230 through the catheter tube 104. In other or further embodiments, the obturator assembly 260 can inhibit or impede blood from flowing through the cannula 241 when the obturator 261 is positioned within the cannula 241, and may be removed from the cannula 241 to permit blood to freely flow through the cannula 241. The obturator assembly 260 thus may function as a valve member that selectively impedes, inhibits, or prevents fluid flow through the cannula 241 or selectively permits fluid flow through the cannula 241. Accordingly, in some embodiments, the obturator assembly 260 may additionally or alternatively be referred to as a valve assembly.

In the illustrated embodiment, the cannula assembly 240 includes a handle 244, which may also or alternatively be referred to as a grasp, grip, flange, tab, or hold. The handle 244 may be fixedly secured to the cannula 240, such as at a proximal end of the cannula 240. In some embodiments, the handle 244 and the cannula 241 may be integrally formed of a unitary piece of material. In other embodiments, the handle 244 may be fixedly secured to the cannula 241, such as by adhesion or overmolding. The handle 244 can be of any suitable form, and may be configured to facilitate introducing the cannula 241 into and through the hub 206 and the placed catheter system 100 and/or to facilitate removal of the cannula 241 therefrom. In the illustrated embodiment, the handle 244 is formed as an elongated grip that extends about a full periphery of the cannula 241. The illustrated handle 244 is formed at a proximal end of the cannula 241, and can be configured to remain outside of and proximal to the hub 206 when the cannula assembly 240 is in a fully inserted orientation relative to the placed catheter system 100. In some embodiments, the lumen 246 of the cannula 241 extends through the handle 244. The handle 244 may be viewed as an extension of the cannula 241, or stated otherwise, the cannula 241 and the handle 244 can form a continuous conduit that includes a continuous lumen 246 therethrough.

In some embodiments, a sealing member 249 may be included within the handle 244. The sealing member 249 can form a fluid seal with the obturator 261 when the obturator 261 is inserted therethrough. In the illustrated embodiment, the sealing member 249 forms a fluid-tight seal with an external surface of the obturator 261 when the access assembly 230 is in the assembled state shown in FIG. 2A. The sealing member 249 may close or seal when the obturator assembly 260 is removed from the cannula assembly 240, thereby closing the proximal end of the lumen 246 and preventing fluid (e.g., blood) from egressing from the proximal end of the cannula 241. Any suitable configuration of the sealing member 249 is contemplated. For example, in some embodiments, the sealing member 249 is formed as a self-sealing polymeric (e.g., silicone) insert.

In certain embodiments, the cannula 241 can include a utility port 243. In the illustrated embodiment, the utility port is positioned at a proximal end or proximal tip of the cannula 243. The utility port 243 may, in some instances, alternatively be referred to as a proximal-most port and/or as an instrument port. In some embodiments, the utility port 243 can be sealed, closed, capped, or otherwise configured or coupled with an element configured to prevent egress of blood or other fluid therefrom. For example, the utility port 243 may be selectively openable so as to permit ingress of one or more instruments therethrough, such as, e.g., the obturator 261, and/or can be selectively closeable or sealable so as to prevent fluid from egressing therefrom after removable of the one or more instruments. For example, in the illustrated embodiment, the utility port 243 is positioned adjacent to the sealing member 249. The sealing member 249 permits the obturator 261 to be advanced therethrough, through the utility port 243 and into and through the cannula 241. Upon removal of the obturator 261, the sealing member 249 can automatically or naturally close, thereby effectively closing the utility port 243 of the cannula 241. As previously discussed, the distal port 247 of the cannula 241 can permit fluid (e.g., blood) to flow into the lumen 246 of the cannula 241 and the proximal port 247 can be configured to permit fluid (e.g., blood) to flow out of the lumen 246 of the cannula 241 when the obturator assembly 260 has been removed. In contrast, blood can be prevented from egressing from the utility port 243 when the obturator assembly 260 is removed, such as by sealing activity of the sealing member 249.

With reference to FIGS. 2A, 2B, 3, and 5, in certain embodiments, the obturator assembly 260 comprises the elongated obturator 261, which can be an elongated rod of any suitable material. The obturator 261 may include a distal tip 263, e.g., a rounded distal tip 263, which may be atraumatic so as not to damage the catheter tube 104 and/or the vessel anatomy when inserted therethrough or therein. The obturator 261 can be formed of any suitable material. In some embodiments, the obturator 261 may be relatively less stiff than the cannula 241. Stated otherwise, at least a distal end of the obturator 261 may be relatively flexible or relatively compliant, as compared with the cannula. In some embodiments, an entirety of the obturator 261 is less stiff, more flexible, or more compliant than the cannula 241. In some embodiments, at least a distal portion of the obturator 261 may be sufficiently flexible to readily track through bends or kinks in the catheter tube 104.

The obturator assembly 260 may include a tab 264, which may also or alternatively be referred to as a grasp, grip, handle, or hold. The tab 264 can be of any suitable form, and may be configured to facilitate introducing the obturator assembly 260 into and through the cannula assembly 240 and/or to facilitate removal of the obturator assembly 260 from the cannula assembly 240. In the illustrated embodiment, the tab 264 is formed as a thin, parallelepiped grippable member that encompasses the proximal end of the obturator 261. The tab 264 is formed at a proximal end of the obturator assembly 260, and can be configured to remain outside of and proximal to the cannula assembly 240 when the cannula assembly 240 is in a fully inserted orientation relative to the hub 206 of the extension set 202.

With reference again to FIGS. 2A and 2B, the access assembly 230 is shown in a fully assembled state and, as previously noted, is shown in an operational stage in which the access assembly 230 has been inserted through the hub 206 and through the base catheter system 100. In this operational state, the sealing member 216 can provide a fluid-tight seal against the cannula 241. The sealing member 216 can be selectively closable. For example, upon removal of the access assembly 230, the sealing member 216 can self-seal to maintain the proximal port of the hub 206 in a sealed or closed state and prevent egress of blood therefrom.

Illustrative examples of certain methods of using the access assembly 230 will now be described. In some embodiments, the hub 206 of the extension set 202 is coupled to the hub 106 of the base catheter system 100. The access assembly 230 may then be inserted through the hub 230 into the base catheter system 100. In particular, the assembled access assembly 230 can be inserted through the proximal port 212 and the chamber 210 of the hub 206, through the hub 106 of the base catheter system 100 and thereby through the distal port 211 of the hub 206, and into and through the lumen 105 of the catheter tube 104. As previously discussed, as the access assembly 230 is advanced through the catheter tube 104, the access assembly 230 can make the catheter tube 104 straighter, smoother, kink-free, and/or otherwise reconfigured to a less tortuous state. In some embodiments, an atraumatic tip 263 of the obturator 261 can inhibit or prevent damage to the catheter tube 104 during insertion of the access assembly 230.

In the illustrated embodiment, and in the operational stage depicted in FIGS. 2A and 2B, a distal end of the access assembly 230 is positioned past a distal tip of the catheter tube 104 when the access assembly 230 is in the fully inserted state. In some instances, advancing the distal end of the access assembly 230 distally beyond the distal tip of the catheter tube 104 may displace a fibrin sheath away from the distal tip of the catheter tube 104 and/or displace a portion of the vessel anatomy, such as a valve, which can permit or facilitate blood withdrawal from the vessel.

In other instances, the distal end of the access assembly 230 may be positioned flush with the distal tip of the catheter tube 104 or may be proximally recessed relative to the distal tip of the catheter tube. For example, in some instances, a fibrin sheath may not be present or may not substantially interfere with blood withdrawal and/or a region distal to the distal tip of the catheter tube 104 may not be obstructed by vessel anatomy (e.g., the vessel wall or a vessel valve structure), and advancing the distal end of the access assembly 230 beyond the distal tip of the catheter tube 104 may not provide any or any significant or substantial advantage for a blood draw. Without being bound by theory, in some embodiments and/or with some patients, advancing the distal end of the access assembly 230 beyond the distal tip of the catheter tube 104 may cause the patient to feel discomfort.

Accordingly, in some embodiments, when the access assembly 230 has been inserted through the catheter tube 104, the distal end of the access assembly 230 can be positioned within a vessel of the patient. The distalmost portion of the access assembly 230 may be directly exposed to the environment within the vessel, or stated otherwise, may extend past the distal tip of the catheter tube 104, or in other embodiments or instances, the distalmost portion of the access assembly 230 may be within the vessel but at least partially covered by the distal end of the catheter tube 104. The distal opening 247 of the cannula 241 can be positioned within and in fluid communication with the interior of the blood vessel.

Once the access assembly 230 is in the desired or predetermined position relative to the catheter tube 104, the obturator assembly 260 may be removed. For example, a practitioner may grip the tab 264 and fully withdraw the obturator assembly 260 proximally from the access assembly 230, thereby leaving the lumen 246 of the cannula 241 empty or open for blood collection. As previously noted, in some embodiments, the obturator assembly 260 substantially inhibits, impedes, or prevents blood flow through the cannula 241 when positioned within the cannula 241, and permits blood flow when removed from the cannula 241. The obturator assembly 260, whether alone or in conjunction with the sealing member 249, may be said to operate as a valve assembly or valve member. When the obturator assembly 260 is fully withdrawn, in some embodiments, the sealing member 249 can close to prevent blood from egressing through the proximal end of the cannula assembly 240.

In some embodiments, a fluid collection device, such as blood collection device, can be coupled to the connector 218. The fluid collection device may provide negative pressure to instigate, enable, conduct, or otherwise effect movement of blood from the vessel, through the distal port 247 of the cannula 241, through the lumen 246 of the cannula 241, through the proximal port 248 of the cannula 241, into and through the cavity 210 of the hub 206, out the side port 213 of the hub 206, through the extension line 217, through the connector 218, and into the fluid collection device. After fluid removal, the fluid collection device can be removed from the connector 218. In some instances, the extension set 202 may be left connected to the placed catheter system 100. In other or further instances, the extension set 202 may be removed after one or multiple blood draws.

In some embodiments, a fluid delivery device (e.g., a syringe) may be coupled with the connector 218 and one or more infusions can be made through the extension set. In some instances, the one or more infusions can be made after each component of the access assembly 230 has been removed from the hub 106. For example, in some embodiments, a saline flush may be performed to clear residual blood from the extension tube 217, the chamber 210 of the hub 207, and the placed catheter system 100 after a blood collection event.

As previously discussed, the cannula 241 can have sufficient structural integrity and/or rigidity to maintain the lumen 246 in an open or patent state, even under negative pressure. The cannula 241 can provide laminar blood flow and/or can avoid turbulent flow or blockages that otherwise can result from a curved and/or kinked catheter tube 104 in the absence of the cannula 241.

In various embodiments, the placed catheter system 100 and/or one or more components of the access system 200 may be included in a kit. For example, the placed catheter system 100 and/or one or more components of the access system 200 can be prepackaged together in packaging that includes instructions for use, which instructions may detail any of the methods disclosed herein.

In some embodiments, the kit may include only the extension set 202 and the access assembly 230. In other embodiments, the extension set 202 and the access assembly 230 may be packaged and/or distributed separately. In some embodiments, the various components of the access system 200 may be prepackaged together in variety of stages of assembly. For example, in some embodiments, the obturator assembly 260 and the cannula assembly 240 may be prepackaged together in a fully assembled state such as depicted in FIG. 2A. In certain of such embodiments, the assembled access assembly 230 may be prepacked with the extension set 202, but fully separated from the extension set 202. In other embodiments, a distal portion of the access assembly 230 may be inserted through the proximal port 212 of the hub 206 and positioned within the chamber 210 of the hub 206 in the prepackaged state. In certain of such embodiments, the proximal exposed portion of the access assembly 230 may be covered with a removable or collapsible sterility covering. Any other suitable packaging or preassembly arrangement is contemplated.

In some embodiments, the extension set 202 may be fixedly secured to the cannula 241. For example, in some embodiments, the sealing member 216 may be omitted and the proximal end of the housing 207 may instead be fixedly attached to the cannula 241 and form a fluid-tight seal therewith. At the distal end of the housing 207, in some embodiments, the connector 208 may be substantially the same as shown in and described with respect to FIG. 2A. In other embodiments, the connector 208 may be omitted. In other or further embodiments, a distal end of the housing 207 may be fixedly attached to the cannula 241 and form a fluid-tight seal therewith. The chamber 210 may be positioned about the proximal port 248 of the cannula 241 and may be fluidly sealed at a position distal to the proximal port 248 and/or at a position proximal to the proximal port 248. In some embodiments, the hub 206 may not be connected to the hub 106 of the preplaced catheter system 100 and/or may be distanced from the preplaced catheter system 100 when the cannula 241 is fully inserted to a desired, predetermined, or delimited position relative to the catheter tube 104. In other embodiments, the hub 206 may form a fluidic seal with the hub 106 but may or may not be securely attached to the hub 106 when the cannula 241 is fully inserted. Other arrangements are contemplated.

In some embodiments, the obturator 261 may be relatively stiffer than at least a portion of the cannula 241. In some (e.g., further) embodiments, the obturator 261 may be internally supportive to the cannula 241 to assist in advancement of the cannula 241 through the preplaced catheter system 100. For example, in some embodiments, the cannula 241 may be relatively more compliant so as to more readily conform to tortuous paths into and through the catheter tube 104, the skin of the patient, and/or the vessel, yet can have sufficient rigidity in at least a radial direction to maintain patency of the lumen 246. In various embodiments, at least a distal end of the cannula 241 may be formed of a polymeric material. For example, the cannula 241 may comprise polyamide. The obturator 261 may comprise a stiffer polymeric material and/or a metallic material (e.g., stainless steel). In some embodiments, the obturator 261, which may be relatively stiffer than at least a proximal end of the cannula 241, may have a distal tip 263 that is flush with or proximally recessed relative to a distal tip of the cannula 241 when in a fully coupled state or when the obturator 261 is fully advanced relative to the cannula 241. In further embodiments, the relatively softer distal tip of the cannula 241 may be relatively atraumatic and may shield the distal tip 263 of the obturator 261 from contact with the catheter tube 104 and/or vasculature of a patient. In other embodiments, the distal tip 263 of a relatively stiffer obturator 261 may extend past the distal tip of the cannula 241. In some embodiments, the coupled obturator 261 and cannula 241 can be advanced into the catheter tube 104 in unison. The obturator 261 can support the relatively softer, less stiff, or more compliant cannula 241 as it is advanced through the catheter tube 104. The obturator 261 can be removed from the cannula 241 after the cannula 241 has been advanced to a desired or predetermined position relative to the catheter tube 104.

As previously discussed, in some embodiments, the cannula 241 is inserted through the base hub 106 and advanced distally through the base catheter tube 104 such that a distal tip of the cannula 241 either extends beyond a distal tip of the base catheter tube 104 and into the bloodstream of the vessel, is flush with the distal tip of the base catheter tube 104, or is proximally recessed relative to the based catheter tube 104. Fluid flow into or from the blood vessel—namely, infusions or aspirations, and blood draws in particular—can be performed through the lumen 246 of the cannula 241. The cannula 241 can be used for functions that might otherwise have been performed directly via the base catheter system 100, such as infusions and/or (e.g., immediately after initial placement) blood collections. Accordingly, the cannula 241 may also be referred to herein as a bypass, superseding, add-on, or transient cannula, whereas the base catheter tube 106 may alternatively be referred to as a pre-placed, placed, or anchor catheter or catheter tube. When the cannula 241 is inserted into the catheter tube 104 to a position suitable for blood collection, the cannula 241 may be said to be in a collection orientation or configuration.

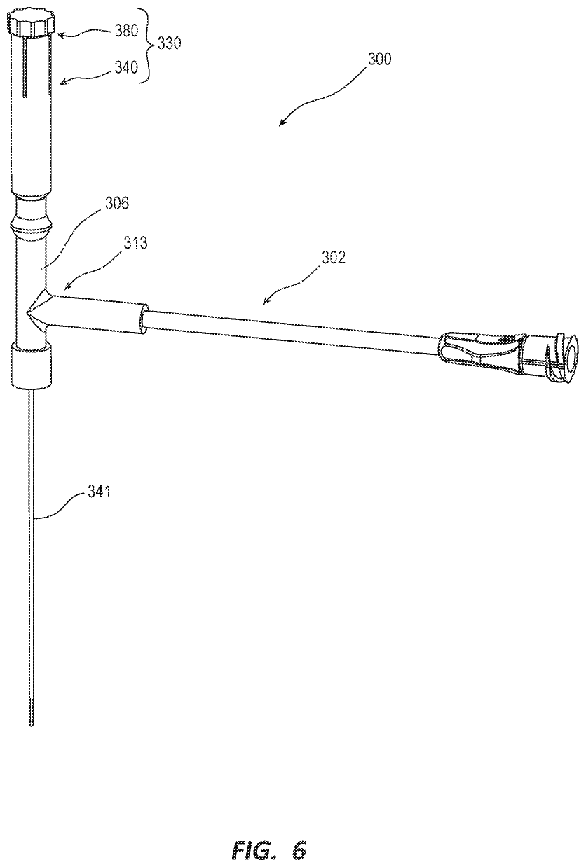

FIG. 6 depicts an embodiment of an access system 300, which can resemble the access system 200 discussed above in many respects. Accordingly, like features of the access systems are designated with like reference numerals, with the leading digits of the access system 300 incremented from “2” to “3.” Relevant disclosure set forth above regarding similarly identified features thus may not be repeated hereafter. Moreover, specific features of the access system 300 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the access system 200 and its various components. Any suitable combination of the features and variations of the same described with respect to the access system 200 can be employed with the access system 300, and vice versa. This pattern of disclosure, and the contemplated combination of any of the described features, applies equally to further embodiments depicted in subsequent figures and described hereafter, wherein the leading digits may be similarly incremented.

The access system 300 can be used with a base catheter system in manners such as previously described. For example, in some embodiments, the access system 300 can be used with a base catheter system such as the base catheter system 100.

The access system 300 can, in some embodiments, include an extension set 302, which can be coupled with the catheter system 100 in any suitable manner. For example, the extension set 302 can include a hub 306 that can be coupled (e.g., securely fastened) to a hub of the catheter system.

The access system 300 can include an access assembly 330 that can be inserted into the base catheter system. In the illustrated embodiment, the access system 300 is advanced through the hub 306 and then through the base catheter system 100 in manners such as previous described.

In the illustrated embodiment, the access assembly 330 includes a cannula assembly 340 and a valve assembly 380. The cannula assembly 340 can include a cannula 341, which can be similar in many respects to the cannula 241. The cannula 341 can, for example, conduct blood from a vessel during a blood collection event. The valve assembly 380 can be selectively manipulated to transition between a closed state, in which the valve assembly 380 prevents fluid communication between a lumen of the cannula 341 and a side port 313 of the extension set 302, and an open state, in which the valve assembly 380 permits fluid communication between the lumen of the cannula 341 and the side port 313.

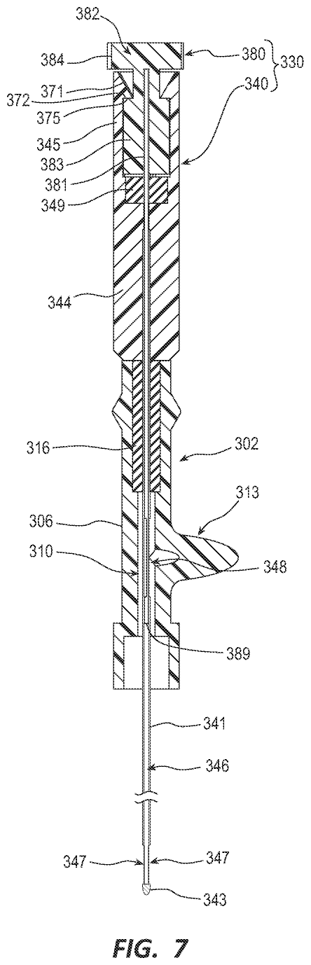

With reference to FIGS. 7 and 9, in the illustrated embodiment, the cannula assembly 340 can further include a handle 344 attached to a proximal end of the cannula 341. The attachment can be of any suitable variety. For example, the cannula 341 may be adhesively joined to the handle 344, such as, for example, in certain embodiments where the cannula 341 is formed of stainless steel.

In some embodiments, the handle 344 can define at least a portion of a coupling feature that retains the cannula assembly 340 and the valve assembly 380 in a coupled arrangement. The coupling feature may further permit relative movement of the cannula assembly 340 and the valve assembly 380. For example, in the illustrated embodiment, the handle 344 includes a coupling feature that retains the valve assembly 380 in a coupled configuration relative to the cannula assembly 340, and further permits selective rotation of the valve assembly 380 relative to the cannula assembly 340.

In the illustrated embodiment, the handle 344 includes a plurality of resilient arms 345. In particular, the handle 344 includes four arms 345 equally spaced about the handle 344. Each arm includes a tapered surface or ramp 371 that is configured to urge the respective arm 345 radially outwardly as the valve assembly 380 is inserted into the handle 344 during assembly. Once the valve assembly 380 has been advanced distally into the handle 340 by a sufficient amount, the arms 345 naturally or resiliently return to an unflexed or unstressed state. Retention surfaces 372 of the arms 345 can hold or retain the valve assembly 380 within the handle 344, as further discussed below. Other embodiments can include different retention mechanisms, and any suitable retention mechanism is contemplated.

With continued reference to FIG. 7, the handle can include a sealing member 349, which can function in a manner such as the sealing member 249 previously discussed. In particular, a valve member 381 of the valve assembly 380 can extend through the sealing member 349. In some embodiments, the sealing member 349 can permit rotation of the valve member 381 therein while maintaining a substantially fluid-tight seal with the valve member 381. The sealing member 349 can prevent blood from egressing from the proximal end of the handle 344 during blood withdrawal.

With reference to FIGS. 7, 8, 11A, and 11B, the cannula 341 can include at least one proximal port 348, which may also be referred to as a side port. The proximal port 348 can be oriented transversely relative to the cannula 341. For example, in the illustrated embodiment, the proximal port 348 extends through a sidewall of the cannula 341. Stated otherwise, the proximal port 348 is positioned at a side of the cannula 341, rather than at an end of the cannula 341, such that a proximal portion of the cannula 341 extends proximally away from the proximal port 348 and a distal portion of the cannula 341 extends distally away from the proximal port 348.

The proximal port 348 can be elongated, in some instances, and may have a length that is substantially greater than an inner diameter of the cannula 341. For example, a length of the proximal port 348 can be greater than an inner diameter of the cannula 341 by a factor of no less than about 1.5, 2.0, 2.5, 3.0, 3.0, 3.5, 4.0, 4.5, or 5.0. In some instances, a relatively long proximal port 348 may encourage laminar flow of blood from the cannula 341 and into and through the hub 306.

In some embodiments, when the cannula 341 is advanced through the hub 306 by a desired amount (e.g., is in a fully advanced orientation), the proximal port 348 can be positioned within a chamber 310 of the hub 306. In some embodiments, the proximal port 348 can be directed toward a side port 313 of the hub. In other embodiments, any suitable relative rotational orientation of the proximal port 348 and the side port 313 may be achieved.

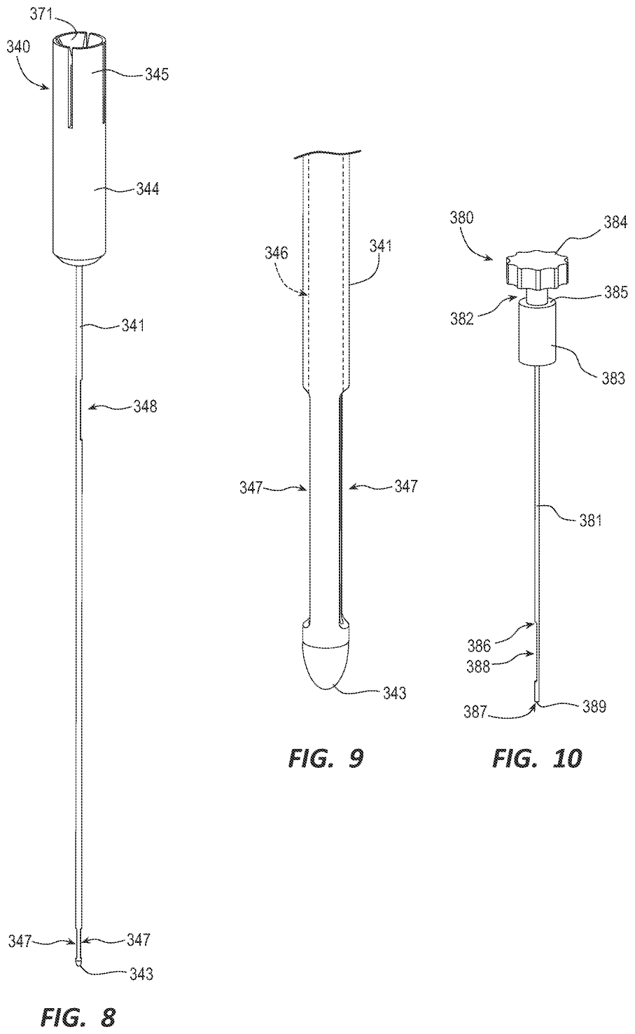

With reference to FIGS. 8 and 9, the cannula 341 can include a distal tip 343 that can resemble the distal end 263 of the obturator 261, in certain respects. For example, in some embodiments, the distal tip 343 can be rounded and can substantially atraumatic, relative to a catheter tube of a preplaced catheter system through which the cannula 341 is advanced. In some embodiments, the cannula 341 can be a metallic (e.g., stainless steel) cannula with a swaged distal tip 343.

The distal end of the cannula 341 can include at least one distal port 347 through which blood can enter into a lumen 346 of the cannula 341 during a blood collection event. In the illustrated embodiment, the cannula 341 includes two distal ports 347. The distal ports 347 are diametrically opposed. Each port 347 is elongated in the longitudinal direction. For example, in some embodiments, a length of each distal port 347 can be greater than an inner diameter of the lumen 346 by a factor of no less than about 1.5, 2.0, 2.5, 3.0, 3.0, 3.5, 4.0, 4.5, or 5.0. In some instances, a relatively long distal port 347 may encourage laminar flow of blood from the vessel into the lumen 346.

In some instances, the illustrated arrangement of distal ports 347 can be advantageous. For example, in some instances, at least a portion of the distal ports 347 may extend distally past a distal tip of the catheter tube within a vessel. In some instances, one of the ports 347 may rest against an anatomical feature of the vessel, such as a sidewall or a valve, and may be blocked thereby. The oppositely oriented port 347 may, however, be unobstructed, such that a blood draw is still possible through the open port 347.

As previously discussed, in some instances, the distal tip 343 of the cannula 341 may be positioned proximally relative to a distal tip of the catheter tube. In some instances, a port 347 may be positioned at the distal tip 343 in addition to or instead of the one or more side ports 347. Any suitable number and arrangement of ports 347 is contemplated.

In the illustrated embodiment, the cannula assembly 340 is not coupled with an obturator, such as other embodiments described herein. In some instances, the cannula 341 may itself achieve certain functionalities that might otherwise be achieved by an obturator, such as by including a rounded distal tip 343 that can lead, guide, and/or advance the cannula 341 atraumatically through the catheter tube. In some instances, the valve assembly 380 can achieve certain functionalities that might otherwise be achieved by an obturator. For example, in some instances, the valve member can selectively prevent or selectively permit blood flow through at least the proximal port 348 of the cannula 341, which may otherwise be accomplished by the presence or absence, respectively, of certain embodiments of an obturator within a cannula.

With reference to FIGS. 7 and 10, in some embodiments, the valve assembly 380 includes the valve member 381, which may also be referred to as a valve shaft 381, valve cannula, valve tube, etc. A proximal end of the valve member 381 can be fixedly secured to a valve body 382. In the illustrated embodiment, the valve body 382 includes a distal portion or plug 383 configured to remain at an interior of the handle 344 and a proximal portion 384 that is accessible at an exterior of the handle 344. The proximal portion 384 may also or alternatively be referred to as a knob, grasp, grip, handle, or hold. As can be seen in FIG. 10, in some embodiments, the proximal portion 384 can include gripping features, which may be positioned about a periphery thereof. The gripping features can facilitate rotation of the valve assembly 380 relative to the cannula assembly 340.

As shown in FIG. 10, in the illustrated embodiment, the plug 383 of the valve body 382 includes a ledge 385 at a proximal end thereof. The ledge 385 interfaces with the retention surfaces 372 of the arms 345 to retain the distal portion 383 within the handle 344, as shown in FIG. 7.

With reference again to FIG. 10, in the illustrated embodiment, the valve member 381 comprises a hollow cannula that defines a lumen 386. The cannula includes a distal opening 387 into the lumen 386. The valve member 381 further includes a side port 388 that extends through a sidewall of the cannula and is in fluid communication with the lumen 386.

Illustrative methods of using the access assembly 300 can substantially resemble methods previously discussed. Rather than removing an obturator to permit blood to flow through the proximal port of the cannula, however, the valve member 381 may be transitioned from a closed state to an open state.

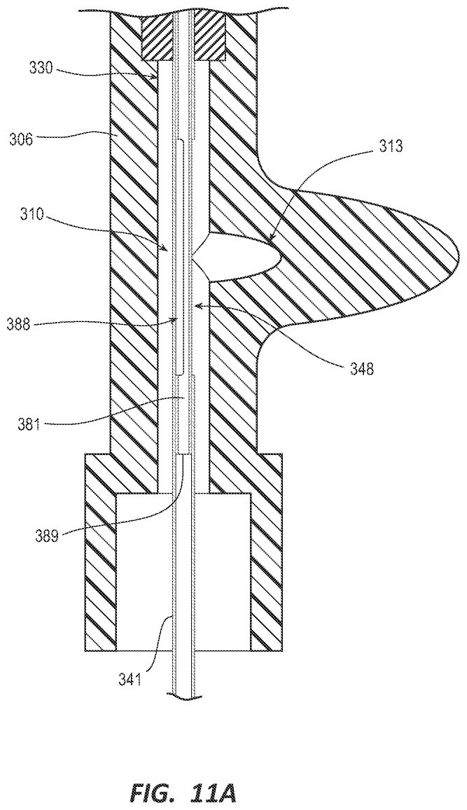

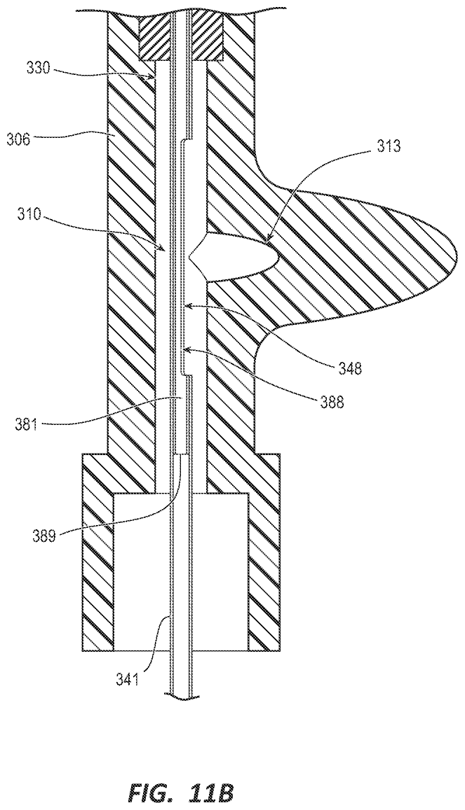

For example, with reference to FIG. 11A, in some embodiments, the access assembly 330 is advanced through the hub 306 when in a closed state. In the closed state, the proximal port 388 of the valve member 381 can be rotated so as to not be aligned with or so as to have no overlap with the proximal port 348 of the cannula 341. Accordingly, blood that may flow into the valve member 381 can be blocked by the sidewall of the valve member 381 so as not to flow through the proximal port 348.

With continued reference to FIG. 11A, in some embodiments, a distal tip 389 of the valve member 381 may extend only a short distance past the proximal port 388. In the illustrated embodiment, the distal tip 389 remains within the hub 306 when the access assembly 330 is fully inserted into the hub 306. Other arrangements are also contemplated. For example, in some embodiments, the valve member 381 may extend distally through a greater portion of the cannula 341.

With reference to FIG. 11B, the valve member 381 can be rotated about a longitudinal axis of the valve member 381 or, more generally, the valve assembly 380 or access assembly 330—e.g., approximately 180 degrees in the illustrated embodiment—to an open state. For example, the valve member 381 can be rotated to align the proximal ports 388, 348 such that the proximal ports permit blood to flow therethrough. The blood can flow into the chamber 310 and out through the side port 313 of the housing 306 in manners such as previously described.

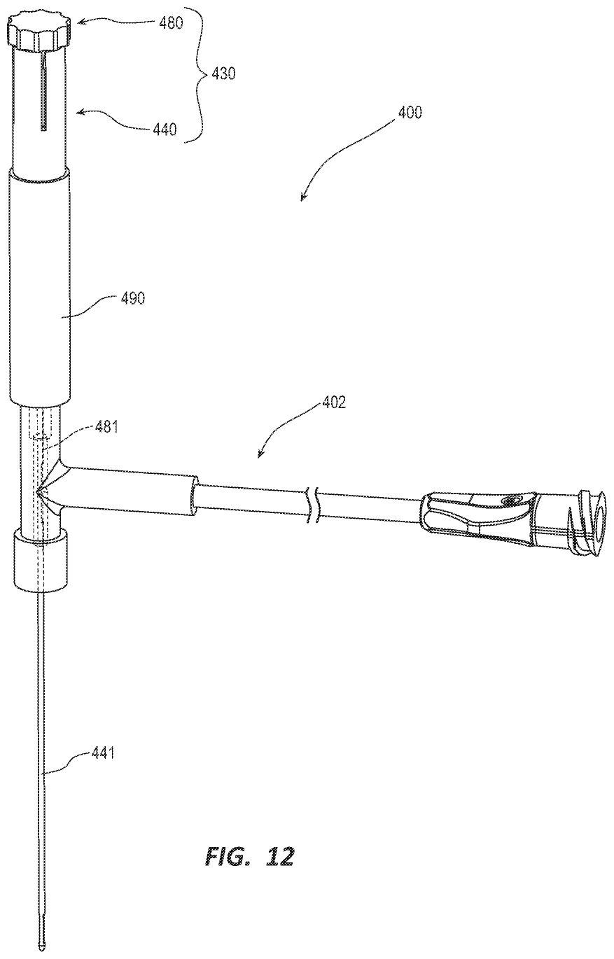

FIG. 12 depicts another embodiment of an access system 400 which resembles other access systems described herein. The access system 400 includes an extension set 402 and an access assembly 430, which resemble like-named and like-numbered features previously described, and further includes a guide 490. The access assembly 430 can include a cannula assembly 440 that includes a cannula 441 and can further include a valve assembly 480 that includes a valve member 481. Certain embodiments of the cannula 441 and the valve member 481 can be relatively longer than certain embodiments of the cannula 341 and the valve member 381 to compensate for the increased length of the system 400 due to the presence of the guide 490.

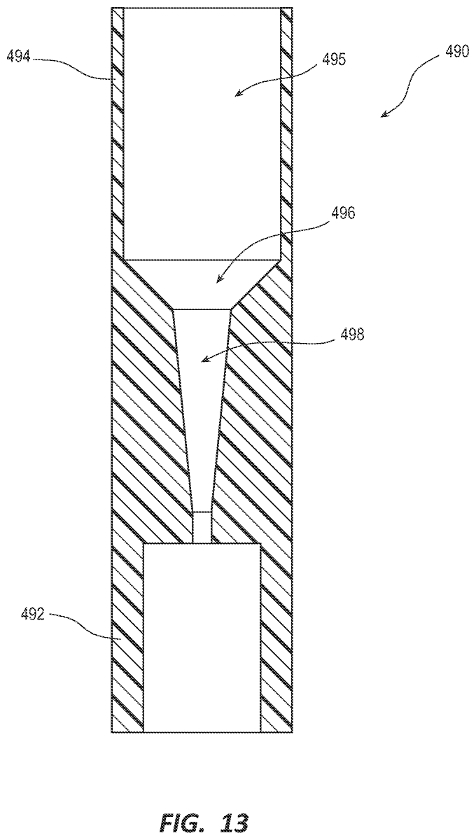

With reference to FIG. 13, the guide 490 can include a distal interface 492 and a proximal interface 494. In some embodiments, the distal interface 492 can be configured to couple with a proximal port of a hub of the extension set 402. For example, the distal interface 492 can include a connector that can securely fasten the guide 490 to the proximal port of hub, such as by a snap-fit, threaded, or other arrangement. In other instances, the distal interface 492 can merely interface with the proximal port of the hub in a nonsecure fashion. In other or further embodiments, the distal interface 492 can be configured to couple directly with a hub of a preplaced catheter system. For example, the guide 490 may be used without an extension set. The distal interface 492 may include a connector to securely connect to the hub of the preplaced catheter system, or may otherwise be configured to interface therewith in a secure or nonsecure fashion.

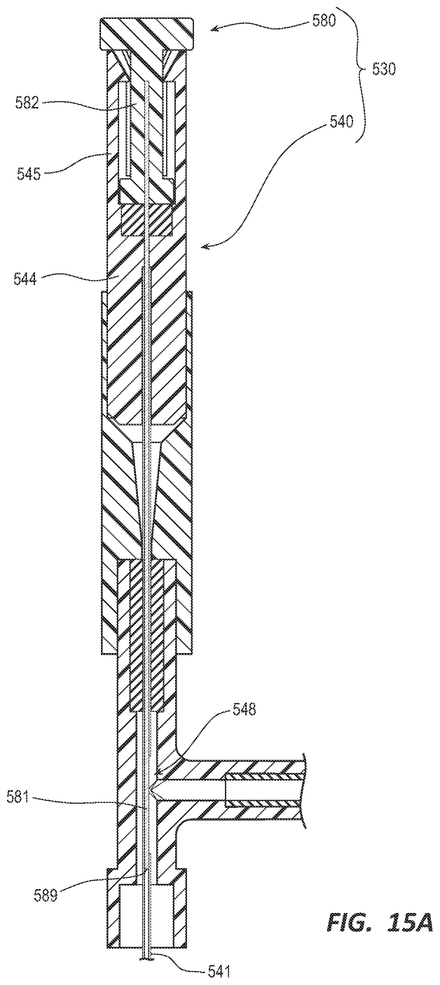

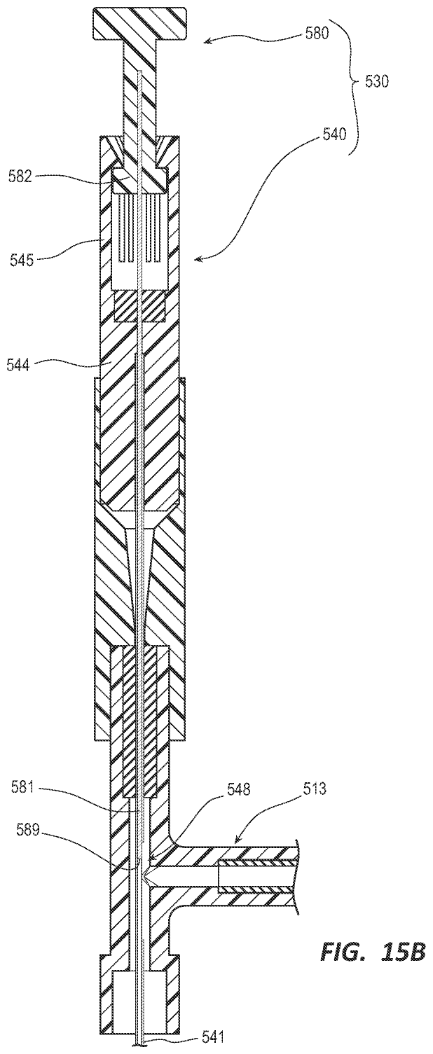

The proximal interface 494 can be configured to receive and/or otherwise interface with the handle portion of the cannula assembly 440. For example, the proximal interface 494 can define a cavity 495 into which at least a distal end of the handle can be received. In some embodiments, a distal end 496 of the cavity 495 may be shaped complementarily to a distal end of the handle for a snug fit. For example, in the illustrated embodiment, the distal end 496 of the cavity 495 is shaped substantially as a frustocone that is complementary in shape to a frustoconical distal end of the handle (see, e.g., the similar arrangement in FIGS. 15A and 15B). In other or further embodiments, the proximal interface 494 may include any suitable keying or other mechanism configured to prevent rotation of the cannula assembly 440 relative to the guide 490.

The guide 490 can further include a guide channel 498 of any suitable variety, which can center, focus, position, or otherwise orient a distal end of the cannula 441 in a desired orientation relative to the hub of the extension set 402 and/or the hub of a preplaced catheter system as the access assembly 430 is advanced distally through the guide 490. In the illustrated embodiment, the guide channel 498 is substantially shaped as a funnel or as a frustocone that tapers inwardly in a distal direction toward a narrowed distal opening.

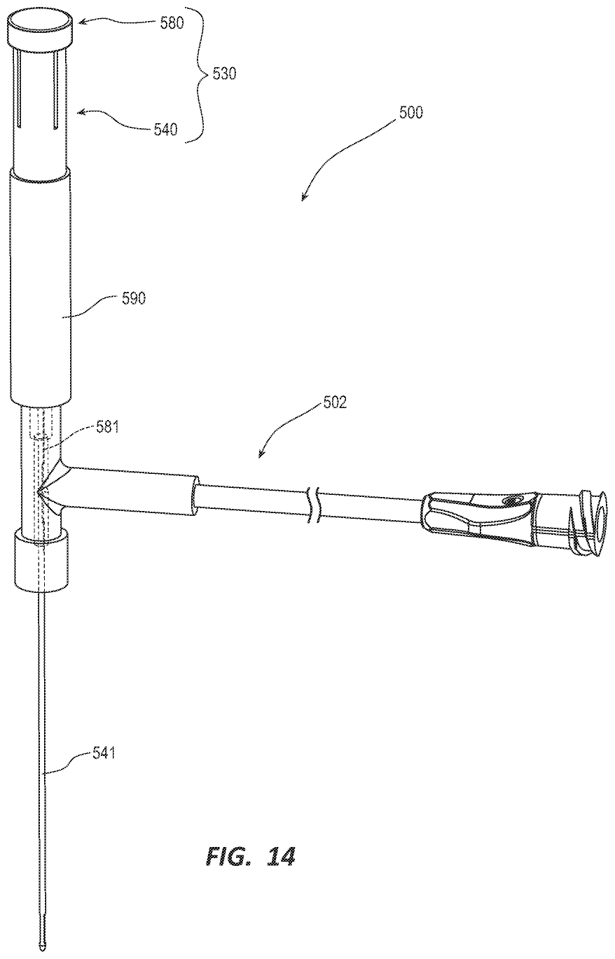

FIG. 14 depicts another embodiment of an access system 500 which resembles other access systems described herein. The access system 500 includes an extension set 502, an access assembly 530, and a guide 590, which resemble like-named and like-numbered features previously described. The access assembly 530 can include a cannula assembly 540 that includes a cannula 541 and can further include a valve assembly 580 that includes a valve member 581. The valve member 581 can be configured to move in at least a longitudinal direction along, e.g., a longitudinal axis of the valve member 581 or, more generally, along a longitudinal axis of the valve assembly 580 or access assembly 530 to transition between closed and open states. In some embodiments, the valve assembly 540 is constrained to translate only or substantially only in the longitudinal direction (e.g., without any or substantially any rotational motion).

As shown in FIG. 15A, a handle 544 can include a plurality of arms 545 that define a cavity within which a distal end of a valve body 582 can be retained and can be free to move at least longitudinally—e.g., distally and proximally. For example, in some embodiments, the valve body 582 can be free to rotate and translate within the handle 544. In other embodiments, the valve body 582 can be constrained to move only or substantially only longitudinally. In the illustrated embodiment, the valve assembly 580 is shown in the closed configuration, with the valve body 582 being positioned at a distal end of the cavity of the handle 544. A distal end of the valve member 581 is positioned distally past a distal end of a side port 548 of the cannula 541. In some embodiments, at least a distal tip 589 of the valve member 581 is solid so as to prevent blood from flowing into the valve member. In any event, the valve member can 581 can prevent blood from flowing between the exterior of the valve member 581 and the interior of the cannula 541 and can prevent blood from flowing out of the side port 548. For example, in some embodiments, an outer surface of the valve member 581 in at least a region at the distal end of the valve member 581 can fluidically seal against an inner surface of the cannula 541. In some embodiments, the distal tip 589 of the valve member 581 is solid such that blood does not flow past the distal tip 589 in a proximal direction. In other embodiments, the valve member 581 may be hollow (e.g., formed as a cannula), such that some blood may flow proximally into the valve member 581 but will be prevented from passing through a sidewall of the valve member 581 and thus prevented from flowing through the side port 548 of the cannula 541.

The valve member 581 may be formed of any suitable material and in any suitable manner. In some embodiments, the valve member 581 comprises a molded or extruded plastic rod.

FIG. 15B depicts the valve assembly 580 in the open configuration. The valve body 582 has been translated longitudinally, retracted, or moved proximally to a proximal end of the cavity of the handle (and is retained within the cavity by the proximal ends of the arms). The distal tip 589 of the valve member 581 is positioned proximal to at least a portion of the side port 548, which thus provides fluid communication between a lumen of the cannula 541 and a side port 513 of the extension set 502.

In some embodiments, the distal tip 589 of the valve member 581 can be tapered or sloped so as to gently direct blood through the side port 548 of the cannula 541 and into the side port 513 of the extension set 502 when the valve assembly 580 is in the open configuration. In some embodiments a sloped distal tip 589 may aid in achieving smooth or laminar blood flow through the side port 548.

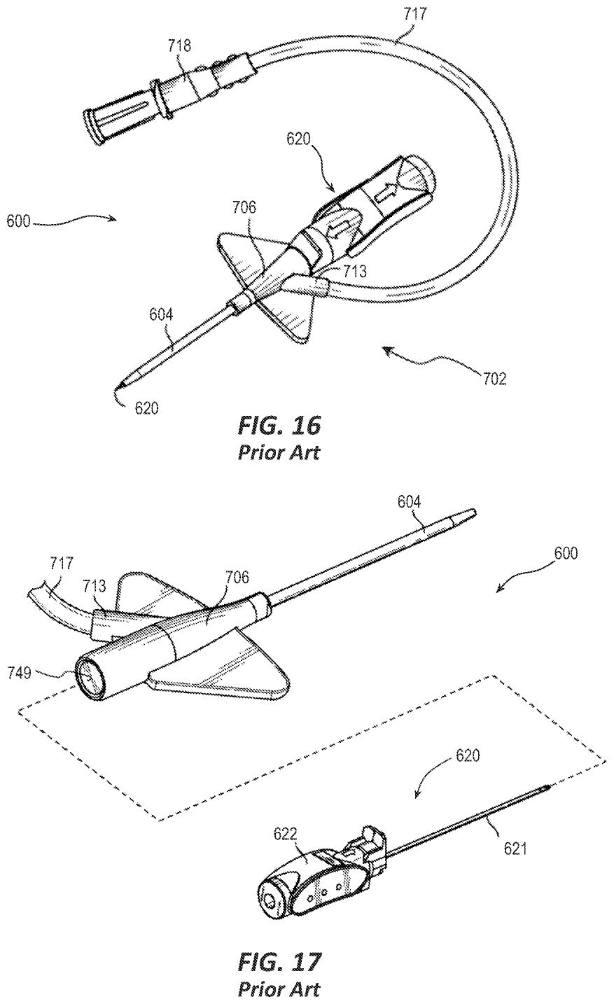

FIG. 16 is a perspective view of an embodiment of a closed catheter system 600, which may also be referred to herein as a closed intravenous catheter system or a closed base catheter system 600. Any suitable closed catheter system is contemplated, such as, for example, any of the NEXIVA™ catheters (e.g., the DIFFUSICS™ line) available from Becton Dickinson. Certain of such closed catheter systems may have an extension set 702 integrated therein. For example, in the illustrated embodiment, a hub 706 of the extension set 702 includes an access port or side port 713 with an extension tube 717 and a connector 718, similar to the extension set arrangements previously disclosed.

The closed catheter system 600 can include a needle assembly 620 that includes a needle 621 connected to a needle hub 622. The needle assembly 620 can be used during insertion of a catheter tube 604 into the vasculature of a patient, and can be removed from the hub 706 thereafter. The needle 621 and its two-part hub 622 can be inserted into the hub 706 and catheter tube 604 during assembly thereof, and can be packaged in such a preassembled configuration, such as that depicted in FIG. 16.

FIG. 17 is a perspective view of the closed catheter system 600 in a disassembled state, such as may be achieved after the catheter tube 604 has been inserted into a blood vessel of a patient. The needle assembly 620 is shown as having been separated from the hub 706 and catheter tube 604. The needle 621 is fixedly attached to the needle hub 622. In the illustrated embodiment, the needle hub 622 is a two-part hub that provides shielding capabilities when the needle 620 is withdrawn from the catheter hub 706 after the catheter tube 604 has been introduced into the vessel of the patient.

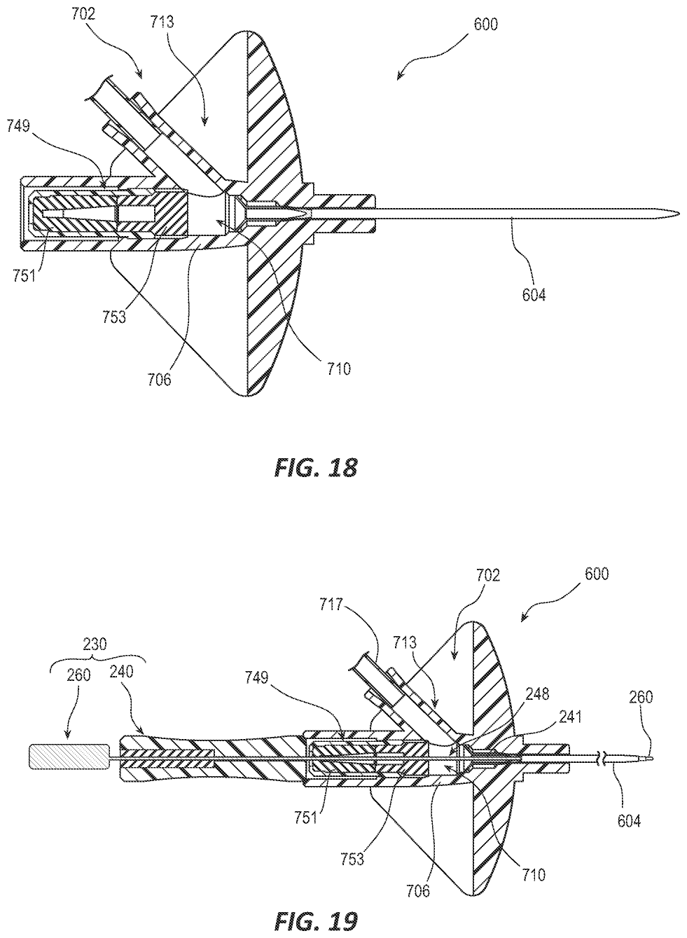

FIG. 18 is a cross-sectional view of the catheter hub and the catheter of the closed catheter system 600 in an operational state in which the needle 621 has been removed from hub 706 and catheter tube 604. The catheter hub 706 can include a distal port in fluid communication with the catheter, a proximal port that includes a sealing member 749, and the side port 713 to which the extension tube and connector are coupled. Each of the ports is in fluid communication with an internal or inner chamber 710. In some embodiments, the sealing member 749 can include a proximal plug 751 and a distal seal 753.

Certain embodiments of access systems, or portions thereof, such as any suitable access system, or portion thereof, disclosed herein (e.g., the access systems 200, 300, 500), can be used with the closed catheter system 600 in this state. In certain embodiments, the access systems may not include their own extension sets, as the catheter hub of the base catheter system already includes an extension set integrated therein, as previously disclosed. For example, only the access assemblies 230, 330, 530 may be used, in some instances. In other or further instances, a guide may be coupled with the closed catheter system 600 and the access assembly (e.g., the access assembly 230, 330, or 530) may be inserted into the closed catheter system 600 via the guide 490.

In some instances, the needle 621 leaves a small, sealed channel through the distal seal 753 of the sealing member 749 when removed therefrom. In certain instances, the distal portion of the access assembly can be inserted through this channel of the sealing member 749. In some embodiments, the closed catheter system 600 is configured for use for power injection. As used herein, “power injection” is consistent with the generally accepted definition of this term, and refers to pressurized infusions that occur at high flow rates, such as up to 4.0 mL/s or up to 5.0 mL/sec; that often involve injection of viscous materials, such as materials (e.g., contrast media) having a viscosity of 11.8 cP+/−0.3 cP; and that take place at elevated pressures. In like manner, a “power injectable” catheter system is one that is capable of sustaining power injection without leaking or bursting. For example, a power injectable catheter or catheter system may be one that complies with the power injection specifications of the International Standards Organization (ISO) standard ISO 10555-1. Thus, for example, embodiments of power injectable closed catheter systems 600 can be configured to sustain power injections. Further embodiments may additionally be used for other functions, such as intravenous therapy at lower pressures or standard infusion and aspiration and/or blood sampling.

In certain embodiments, an access assembly 230, 330, or 530 may be used to sample blood from a patient via the closed catheter system 600 without affecting power injectability of the catheter system 600. For example, in some embodiments, the distal seal 753 of the sealing member 749 may be pierced by the cannula of an access assembly for blood sampling, the cannula may be removed from the sealing member 749 after the blood collection event, and power injection via the side port 713 may still be possible thereafter. In some embodiments, multiple such blood sampling events using a series of individually inserted and removed cannulae are possible. For example, in some embodiments, an obturator helps preserve the sealing member 749. In other embodiments, a suaged tip of a cannula helps preserve the sealing member 749.

FIG. 19 is a cross-sectional view of an embodiment of the access assembly 230 in an assembled stated and positioned within the hub 706 of the closed catheter system 600. In some embodiments, the cannula inserted through the catheter tube 604 can straighten the catheter tube 604, such as in manners previously disclosed with respect to the catheter tube 104. Once in a desired or predetermined position, the obturator assembly 260 may be removed to open the lumen 246 of the cannula 241 (see also FIG. 2A). The proximal port 248 of the cannula 241 can be positioned within the open chamber 710 of the catheter hub 706. Blood thus can enter into the cannula 241 at the distal end thereof, pass through the lumen of the cannula 241, through the side port/proximal port 248 of the cannula that is positioned within the chamber 710, through the chamber 710, through the side port 713 of the catheter hub 706, through the extension tube 717, through the connector 718 (FIG. 16), and into the fluid collection device. After a blood collection event, the cannula 241 can be removed from the closed catheter system 600.

Any suitable combination of the various features of the various embodiments disclosed herein is contemplated. For example, some embodiments of access systems that are described without the presence of a guide (such as the guides 490, 590) may include a guide, and vice versa. As a further example, in some embodiments of access systems that are described herein as including an extension sets, the extension set may be omitted in some instances.

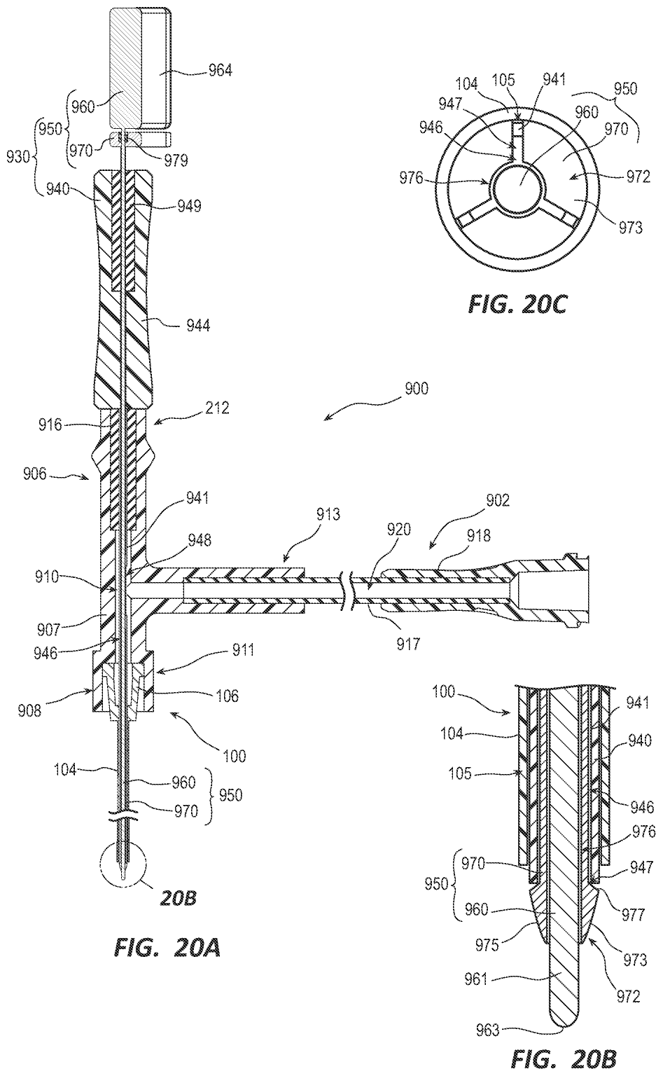

With reference to FIGS. 20A-20C, in some embodiments, another embodiment of an access system 900 can be configured to couple with certain open catheter systems, such as, for example, the base catheter system 100. As further discussed below, other or further embodiments may alternatively or additionally be configured to couple with certain closed catheter systems, such as the closed catheter system 600. The access system 900 can also be referred to as a fluid channeling system, a fluid extraction system, a blood removal system, a follow-on cannula system, etc.

The base catheter system 100, such as the catheter tube 104 thereof, can be inserted into a vessel of a patient in any suitable manner, such as previously described. One or more portions of the access system 900 may be coupled to the base catheter system 100. In some instances, no portion of the access system 900 is coupled with the base catheter system 100 prior to insertion of the base catheter system 100 into the patient. In other instances, at least a portion of the access system 900 (such as, e.g., an extension set) may be coupled with the base catheter system 100 prior to insertion of the base catheter system 100 into the vasculature of the patient.

FIGS. 20A-20C show the illustrated access system 900 fully coupled with the base catheter system 100. For example, FIGS. 20A-20C can be illustrative of a stage of use of the access system 900 at a time after the base catheter system 100 has been inserted into the vasculature of the patient and after the access system 900 has been coupled with the base catheter system 100, but before fluid is withdrawn from the vessel of the patient via the access system 900.

In the illustrated embodiment, the access system 900 includes an extension set 902 attached to the hub 106 of the base catheter system 100. The extension set 902 can include a hub 906 that includes any suitable connector 908 configured to interface with the hub 106 of the base catheter system 100 and thereby connect the extension set 902 to the hub 106. For example, in some embodiments, the connector 908 can comprise a luer connector, a luer lock connector, or any other suitable connector, which may provide a fluid seal with the hub 106. For example, in some embodiments, the hub 106 is formed as a female luer, and the connector 908 is formed as a male luer or as a locking male luer.

In various embodiments, the hub 906 may include a handle or housing 907. In the illustrated embodiment, the housing 907 of the hub 906 defines an open interior, cavity, or internal chamber 910. As discussed hereafter, the chamber 910 may permit fluid flow therethrough, such as to permit blood that has egressed from the vessel to pass therethrough during a blood withdrawal and/or to permit saline to pass therethrough in a substantially opposite direction during flushing. The hub 906 may include a first port 911, a second port 912, and a third port 913, each of which may be in fluid communication with the chamber 910.

In the illustrated embodiment, the first port 911 is positioned at a distal end of the hub 906, and may be referred to as a distal port or as a connection port. The connector 908 may be positioned at or about the first port 911. Blood from the vessel may enter the hub 906 through the first port 911 and/or infused fluid may exit from the hub 906 through the first port 911.

In the illustrated embodiment, the second port 912 is positioned at a proximal end of the hub 906. The second port 912 may be referred to as a proximal port or as a valved port. In the illustrated embodiment, the second port 912 includes a valve, septum, or sealing member 916. As further discussed below, various components of the access assembly 900 may be introduced through and/or withdrawn from the second port 912. The sealing member 916 can prevent fluid from escaping from the chamber 910 via the second port 912.

The third port 913 may be referred to as an access port. In the illustrated embodiment, the third port 913 is positioned at a lateral position relative to a longitudinal axis of the hub 906. The third port 913 may alternatively be referred to as a side port. The third port 913 may permit blood withdrawals, infusions of any suitable variety, and/or saline flushes therethrough. Stated otherwise, blood obtained from the vessel may exit from the hub 906 through the third port 913 and/or infusion fluids may enter the hub 906 through the third port 913.

In some embodiments, an extension tube 917 is connected to the third port 913. Any suitable tubing is contemplated. The extension tube 917 can be attached at a proximal end thereof with any suitable connector 918. The connector 918 can be configured to couple with any suitable fluid source and/or fluid collection device or may, in other embodiments, be integrally formed with fluid source and/or fluid collection apparatus. For example, in the illustrated embodiment, the connector 918 is formed as female luer connector configured to couple with a syringe, which may be used for infusion or blood collection. The connector 918 may be used for connection to blood draw apparatus, such as syringes or evacuated blood collection tubes (e.g., Vacutainer® tubes available from Becton Dickinson) and/or apparatus therefor. For example, in some embodiments, the connector 918 may include or may be configured to connect with a Vacutainer® one-use holder, via which blood draws may be made into one or more Vacutainer® tubes.

In the illustrated embodiment, the connector 918 does not provide any valving relative to a lumen 920 that extends through the extension tube 917. In some instances, a clamp, such as a standard sliding clamp or a standard thumb-actuated compression clamp (not shown) may selectively prevent or permit fluid flow through the extension tube 917. In other embodiments, the connector 918 may comprise a valve and/or a clamp may be omitted. For example, in some embodiments, the connector 918 comprises a needleless connector valve of any suitable variety.

The access system 900 can include an access assembly 930. In the illustrated embodiment, the access assembly 930 includes a cannula assembly 940 that includes a cannula 941, which may also be referred to as a fluid channeler, a fluid extraction member, a thin-walled cannula, etc., and an obturator assembly 950, which may also be referred to as an obturator. In the illustrated embodiment, the obturator assembly 950 includes a core 960 and a guide member or spacer 970. The obturator assembly 950 may be referred to as a multi-part obturator. In other embodiments, the obturator assembly 950 may comprise a single component. For example, in some embodiments, the obturator assembly 950 may include only the core 960, which may itself be referred to as an obturator.

The cannula 941 can comprise a thin-walled member of formed of any suitable material. For example, in various embodiments, the cannula 941 comprises a metallic tube (e.g., stainless steel) or a plastic tube (e.g., extruded plastic). In some embodiments, the cannula 941 is relatively rigid or is less laterally flexible than the catheter tube 104. The cannula 941 can provide structural support to the catheter tube 104 when inserted therein, or stated otherwise, can itself maintain a lumen 946 that passes through the cannula 941 in an open configuration when the cannula 941 is inserted through the catheter tube 104. In some embodiments, the cannula 941 can be sufficiently rigid to maintain a substantially rectilinear orientation when inserted through the catheter tube 104 when placed in the patient such that, even when positioned through the skin and into a vessel of the patient, the cannula 941 is substantially rectilinear. In other embodiments, the cannula 941 may be sufficiently rigid so as to curve only slightly, or may be said to curve or bend substantially less than the catheter tube 104 while extending through the skin and into the vessel of the patient. Stated otherwise, the cannula 941 may straighten, render less curved or angled, unkink, or otherwise reconfigure the catheter tube 104 to be more rectilinear when the cannula 941 is inserted through at least a portion of the lumen 105 of the catheter tube 104. In various instances, the relatively straightened, less curved, and/or unkinked configuration of the cannula 941 when extending through the place catheter tube 104 can provide a laminar flow of blood through the lumen 946 of the cannula 941, which can provide a better blood sample. For example, blood samples obtained through the cannula 941 may be free or relatively free of hemolysis.

The cannula 941 can include a distal opening or distal port 947 at a distal end of the cannula 941 that is in fluid communication with the lumen 946. In the illustrated embodiment, the distal port 947 is positioned at the distalmost tip of the cannula 941. In other embodiments, the port 947, or one or more additional ports 947, can be positioned at the side of the distal tip of the cannula 941, such as in a lateral orientation and/or proximal to the distal tip.

The cannula 941 can further include a proximal opening or proximal port 948 at a proximal portion of the cannula 941 that is in fluid communication with the lumen 946. In the illustrated embodiment, the proximal port 948 is relatively elongated and is positioned at a side of the cannula 941. The port 948 may be said to extend through a sidewall of the cannula 941. The port 948 may also be referred to as a side port. In the illustrated embodiment, the proximal port 948 is positioned such that when the cannula 941 is inserted (e.g., fully inserted) into or through the catheter tube 104, such as in a manner that puts the distal port 947 in fluid communication with the interior of the vessel of the patient, the proximal port 948 is positioned within the chamber 910 of the hub 906. Accordingly, blood may enter through the distal port 947, pass through the lumen 946, and egress through the proximal port 948 into the chamber 910 of the hub 906 during blood collection. The blood may then pass through the third or side port 913 of the hub, through the extension tube 917, and through the connector 918 into whatever blood collection device has been coupled with the connector 918. In other embodiments, the cannula 941 can include a plurality of proximal ports 948.

In some embodiments, the access assembly 930 may only be comprised of the cannula assembly 940. For example, the cannula 941 of the cannula assembly 940 may be inserted through the hub 906 and through the placed catheter system 100, e.g., to collect blood. The cannula 941 may subsequently be removed from the hub 906.

In other embodiments, the obturator assembly 950 may be used with the cannula 941, in manners such as described hereafter. The obturator assembly 950 can, in some embodiments, assist inhibit or prevent the distal tip of the cannula 941 from scraping, piercing, tearing, or otherwise damaging the catheter tube 104 during insertion of the access assembly 930 through the catheter tube 104.

In the illustrated embodiment, the cannula assembly 940 includes a handle 944, which may also or alternatively be referred to as a grasp, grip, flange, tab, or hold. The handle 944 may be fixedly secured to the cannula 940, such as at a proximal end of the cannula 940. In some embodiments, the handle 944 and the cannula 940 may be integrally formed of a unitary piece of material. The handle 944 can be of any suitable form, and may be configured to facilitate introducing the cannula 941 into and through the hub 906 and the placed catheter system 100 and/or to facilitate removal of the cannula 941 therefrom. In the illustrated embodiment, the handle 944 is formed as an elongated tab or flange that extends about a full periphery of the cannula 941. The illustrated handle 944 is formed at a proximal end of the cannula 941, and can be configured to remain outside of and proximal to the hub 906 when the cannula assembly 940 is fully inserted into the placed catheter system 100. In some embodiments, the lumen 946 of the cannula 941 extends through the handle 944.

In some embodiments, a sealing member 949 may be included within the lumen 946, such as, for example, within the handle region of the lumen 946, to form a fluid seal with the obturator assembly 930. In the illustrated embodiment, the sealing member 949 forms a fluid-tight seal with an external surface of the spacer 970 when the access assembly 930 is in the assembled state shown in FIG. 20A. The sealing member 949 may close or seal when the obturator assembly 930 is removed from the cannula 941, thereby closing the proximal end of the lumen 946 and preventing fluid (e.g., blood) from egressing from the proximal end of the cannula 941. Any suitable sealing member is contemplated.

In certain embodiments, the core 960 comprises an elongated rod 961 with a rounded distal tip. The distal tip 963 may be atraumatic so as not to damage the catheter tube 104 and/or the vessel anatomy when inserted therethrough or therein. The core 960 can be formed of any suitable material. In some embodiments, the core 960 may be relatively less stiff than the cannula 941. Stated otherwise, the core 960 may be relatively flexible. For example, at least a distal end of the core 960 may be sufficiently flexible to readily track through bends or kinks in the catheter tube 104.

The core 960 may include a tab 964, which may also or alternatively be referred to as a grasp, grip, flange, handle, or hold. The tab 964 can be of any suitable form, and may be configured to facilitate introducing the core 960 into and through the spacer 970 and/or to facilitate removal of the core 960 from the spacer 970. In the illustrated embodiment, the tab 964 is formed as a flange that extends about a full periphery of the core 960. The tab 964 is formed at a proximal end of the core 960, and can be configured to remain outside of and proximal to the spacer 970 when the core 960 is fully inserted into the spacer 970.

The spacer 970 can include at least a distal end that is configured to expand and contract. In the illustrated embodiment, the distal end expands laterally outward when the core 960 is inserted therethrough. In some embodiments, the distal end of the spacer 970 can be biased radially inwardly such that the distal end automatically contracts radially inwardly when the core 960 is removed from the spacer 970. In other embodiments, the distal end of the spacer 970 can be biased toward or otherwise may naturally be in the expanded state. The distal end of the spacer 970 thus may be urged to a contracted state for removal from the access assembly 930, such as in a manner discussed below.

In the illustrated embodiment, the spacer 970 includes a guide head 972 that is selectively expandable and contractable, such as in any of the manners just described. In the illustrated embodiment, the guide head 972 includes three collet members 973. The illustrated collet members 973 are equally sized and are spaced at regular angular intervals (e.g., each collet member 973 defines an arc of approximately 120 degrees).

In some instances, during assembly of the access assembly 930, the guide head 972 can be advanced through the cannula 941 and then distally past the distal tip of the cannula 941 while in a constricted or contracted state. The core 960 can be inserted distally through a lumen 976 defined by the spacer 970. As the core 960 passes through the guide head 972, the guide head 972 can be expanded to the expanded state shown in FIGS. 20A-20C. In the illustrated embodiment, the core 960 extends past a distal tip of the spacer 970.

In the illustrated embodiment, the guide head 972 can define an outer perimeter that is at least as large as an outer perimeter of the cannula 941. Stated otherwise, in various embodiments, the guide head 972 can shield a distal face and or distal edge of the cannula 941. The guide head 972 can guide the cannula 941 through the catheter tube 104 and can prevent or inhibit the distal tip of the cannula 941 from contacting the catheter tube 104 during insertion of the access assembly 930 through the catheter tube 104. The guide head 972 can be tapered inward in a proximal-to-distal direction, which can facilitate passage of the access assembly 930 through kinks, curves, or other tortuous pathways defined by the catheter tube 104. For example, in the illustrated embodiment, each collet member 973 includes a distal surface 975 that defines a shallow angle relative to a longitudinal axis of the access assembly 930.

In the illustrated embodiment, a proximal portion of the guide head 972 is inwardly tapered in the distal-to-proximal direction. That is, a proximal surface 977 of each collet member 973 of the guide head 972 can be tapered or angled. In some instances, such as when the guide head 972 is biased to the low-profile or radially contracted orientation, the tapered proximal surface 977 can aid in ensuring that the guide head 972 is expanded cover (e.g., as viewed end-on, such as in FIG. 20C) large portions or all of the distal tip of the cannula 941 as the core 960 is advanced distally through the spacer 970. In other or further embodiments, the proximal surface 977 can assist in transitioning the guide head 972 to the contracted orientation as the spacer 970 is moved proximally relative to the cannula 941. For example, in either outwardly biased or inwardly biased embodiments, the proximal surface 977 can interact with the inner edge of the distal tip of the cannula 941 as the spacer 970 is moved proximally relative to the cannula 941 to force the collet members 973 radially inward to a contracted orientation suitable for passing proximally through the cannula 941.

In the illustrated embodiment, the spacer 970 includes a tab 974 at a proximal end thereof, which may also or alternatively be referred to as a grasp, grip, flange, handle, or hold. The tab 974 can be of any suitable form, and may be configured to facilitate introducing the spacer 970 into and through the cannula 941 and/or to facilitate removal of the spacer 970 from the cannula 941. In the illustrated embodiment, the tab 974 is formed as a flange that extends about a full periphery of the spacer 970. The tab 974 is formed at a proximal end of the spacer 970, and can be configured to remain outside of and proximal to the cannula 941 when the spacer 970 is fully inserted into the cannula 941. In some embodiments, a lumen 976 of the spacer 970 extends through the tab 974.

In some embodiments, a sealing member 979 may be included within the lumen 976, such as, for example, within the tab region of the lumen 976, to form a fluid seal with the core 960. The sealing member 979 may close or seal when the core 960 is removed from the spacer 970, thereby closing the proximal end of the lumen 976 and preventing fluid (e.g., blood) from egressing from the proximal end of the spacer 970. Any suitable sealing member is contemplated.

In FIGS. 20A-20C, the access assembly 930 is shown in a fully assembled state and, as previously noted, is shown in an operational stage in which the access assembly 930 has been inserted through the hub 906 and through the base catheter system 100. Illustrative examples of methods in which such an arrangement may be achieved for one or more stage of the methods will now be described.

In some embodiments, the hub 906 of the extension set 902 is coupled to the hub 106 of the base catheter system 100. The access assembly 930 may then be inserted through the hub 906 of the extension set 902 and then into the base catheter system 100. In particular, the assembled access assembly 930 can be inserted through the proximal port 912 and the chamber 910 of the hub 906, through the hub 106 of the base catheter system 100 and thereby through the distal port 911 of the hub 906, and into and through the lumen 105 of the catheter tube 104. As previously discussed, as the access assembly 930 is advanced through the catheter tube 104, the access assembly 930 can make the catheter tube 104 straighter, smoother, kink-free, and/or otherwise reconfigured to a less tortuous state.

In the illustrated embodiment, and in the operational stage depicted in FIGS. 20A-20C, a distal end of the access assembly 930 is positioned past a distal tip of the catheter tube 104 when the access assembly 930 is in the fully inserted state. In some instances, advancing the distal end of the access assembly 930 distally beyond the distal tip of the catheter tube 104 may displace a fibrin sheath away from the distal tip of the catheter tube 104 and/or displace a portion of the vessel anatomy, such as a valve, which can permit or facilitate blood withdrawal from the vessel.

In other instances, the distal end of the access assembly 930 may be positioned flush with the distal tip of the catheter tube 104 or may be proximally recessed relative to the distal tip of the catheter tube. For example, in some instances, a fibrin sheath may not be present or may not substantially interfere with blood withdrawal and/or a region distal to the distal tip of the catheter tube 104 may not be obstructed by vessel anatomy (e.g., the vessel wall or a vessel valve structure), and advancing the distal end of the access assembly 930 beyond the distal tip of the catheter tube 104 may not provide any or any significant or substantial advantage for a blood draw. Without being bound by theory, in some embodiments and/or with some patients, advancing the distal end of the access assembly 930 beyond the distal tip of the catheter tube 104 may cause the patient to feel discomfort.

Accordingly, in some embodiments, when the access assembly 930 has been inserted through the catheter tube 104, the distal end of the access assembly 930 can be positioned within a vessel of the patient. The distalmost portion of the access assembly 930 may be directly exposed to the environment within the vessel, or stated otherwise, may extend past the distal tip of the catheter tube 104, or in other embodiments or instances, the distalmost portion of the access assembly 930 may be within the vessel but at least partially covered by the distal end of the catheter tube 104.

Once the access assembly 930 is in the desired or predetermined position relative to the catheter tube 104, the core 960 may be removed. For example, a practitioner may grip the tab 964 and fully withdraw the core 960 proximally from the access assembly 930, thereby leaving the lumen 976 empty and providing space for contraction of the guide head 972. When the core 960 is fully withdrawn, in some embodiments, the sealing member 979 can close to prevent blood from egressing through the proximal end of the spacer 970.

The spacer 970 may then be withdrawn from the cannula 941. In some embodiments, removal of the core 960 may cause the guide head 972 to automatically contract inwardly to a low-profile state, such as due to a bias of the collet members 973, to permit removal of the spacer 970. In other or further embodiments, the guide head 972 may contract inwardly to permit removal as the spacer 970 is withdrawn proximally, in manners such as previously described. The spacer 970 may be fully withdrawn from the cannula 941. When the spacer 970 is fully withdrawn, in some embodiments, the sealing member 949 can close to prevent blood from egressing through the proximal end of the cannula 941. Removal of the core 960 and the spacer 970, otherwise referred to as the obturator assembly 950, can leave the lumen 946 of the cannula 941 open for blood collection.

In some embodiments, a fluid collection device, such as blood collection device, can be coupled to the connector 918. The fluid collection device may provide negative pressure to instigate, enable, conduct, or otherwise effect movement of blood from the vessel, through the distal port 947 of the cannula 941, through the lumen 946 of the cannula, through the proximal port 948 of the cannula, into and through the cavity 910 of the hub 906, out the side port 913 of the hub 906, through the extension line 917, through the connector 918, and into the fluid collection device. After fluid removal, the fluid collection device can be removed from the connector 918. In some instances, the extension set 902 may be left connected to the placed catheter system 100. In other or further instances, the extension set 902 may be removed after one or multiple blood draws.

In some embodiments, a fluid delivery device (e.g., a syringe) may be coupled with the connector 918 and one or more infusions can be made through the extension set. In some instances, the one or more infusions can be made after each component of the access assembly 930 has been removed from the hub 106. For example, in some embodiments, a saline flush may be performed to clear residual blood from the extension tube 917, the chamber of the hub 907, and the placed catheter system after a blood collection event.

As previously discussed, the cannula 941 can have sufficient structural integrity and/or rigidity to maintain the lumen 946 in an open or patent state, even under negative pressure. The cannula 941 can provide laminar blood flow and/or can avoid turbulent flow or blockages that otherwise can result from a curved and/or kinked catheter tube 104 in the absence of the cannula 941.

In various embodiments, the placed catheter system 100 and/or one or more components of the access system 900 may be included in a kit. For example, the placed catheter system 100 and/or one or more components of the access system 900 can be prepackaged together in packaging that includes instructions for use, which instructions may detail any of the methods disclosed herein.

In some embodiments, the kit may include only the extension set 902 and the access assembly 930. In other embodiments, the extension set 902 and the access assembly 930 may be packaged and/or distributed separately. In some embodiments, the various components of the access system 900 may be prepackaged together in variety of stages of assembly. For example, in some embodiments, the core 960, the spacer 970, and the cannula 941 may be prepackaged together in a fully assembled state such as depicted in FIG. 20A. In certain of such embodiments, the assembled access assembly 930 may be prepacked with the extension set 902, but fully separated from the extension set 902. In other embodiments, a distal portion of the access assembly 930 may be inserted through the proximal port 912 of the hub 906 and positioned within the chamber 910 of the hub 906 in the prepackaged state. In certain of such embodiments, the proximal exposed portion of the access assembly 930 may be covered with a removable or collapsible sterility covering. Any other suitable packaging or preassembly arrangement is contemplated.

In some embodiments, the extension set 902 may be fixedly secured to the cannula 941. For example, in some embodiments, the sealing member 916 may be omitted and the proximal end of the housing 907 may instead be fixedly attached to the cannula 941 and form a fluid-tight seal therewith. At the distal end of the housing 907, in some embodiments, the connector 908 arrangement may be substantially the same as shown in and described with respect to FIG. 20A. In other embodiments, the connector 908 may be omitted. In other or further embodiments, a distal end of the housing 907 may be fixedly attached to the cannula and form a fluid-tight seal therewith. The chamber 910 may be positioned about the proximal port 948 of the cannula 941 and may be fluidly sealed at or at a position distal to the proximal port 948 and may further be fluidly sealed at or at a position proximal to the proximal port 948. In some embodiments, the hub 906 may not be connected to the hub 106 of the preplaced catheter system 100 and/or may be distanced from the preplaced catheter system 100 when the cannula 941 is fully inserted to a desired, predetermined, or delimited position relative to the catheter tube 104. In other embodiments, the hub 906 may form a fluidic seal with the hub 106 but may or may not be securely attached to the hub 106 when the cannula 941 is fully inserted. Other arrangements are contemplated.

As previously discussed, in some embodiments, the cannula 941 is be inserted through the base hub 106 and advanced distally through the base catheter tube 104 such that a distal tip of the cannula 941 extends beyond a distal tip of the base catheter tube 104 and into the bloodstream of the vessel, is flush with the distal tip of the base catheter tube 104, or is proximally recessed relative to the based catheter tube 104. Fluid flow into or from the vessel—namely, infusions or aspirations, and blood draws in particular—can be performed through the lumen 946 of the cannula 941. The cannula 941 may can be used for functions that might otherwise have been performed directly via the base catheter system 100. Accordingly, the cannula 941 may also be referred to herein as a bypass, superseding, add-on, or transient cannula, whereas the base catheter tube 106 may alternatively be referred to as a pre-placed, placed, or anchor catheter or catheter tube.

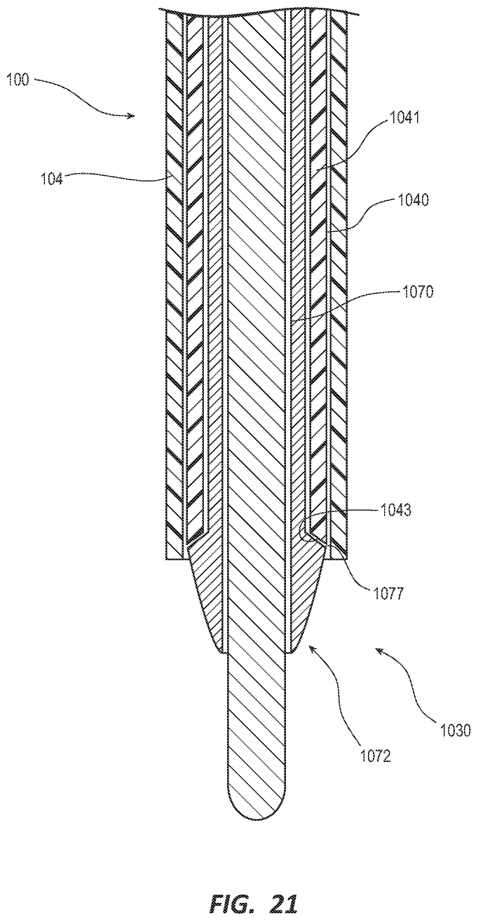

FIG. 21 depicts an embodiment of an access assembly 1030 positioned within the base catheter system 100. The illustrated access assembly 1030 includes a guide member 1070 with a guide head 1072 that resemble the spacer 970 and the guide head 972 discussed above, and can include one or more angled proximal surfaces 1077. The access assembly 1030 further includes a cannula assembly 1040 that includes a cannula 1041 having a tapered distal tip 1043. In the illustrated embodiment, the distal tip 1043 includes an internal taper that is shaped substantially as a frustocone. An angle of the internal taper or bevel may match or substantially match an angle of the proximal surface 1077. During insertion, with the access assembly 1030 in a fully assembled state, the distal tip 1043 may abut against or otherwise closely conform to the proximal surfaces 1077, which can avoid any gaps into which the catheter tube 104 could get caught. In other or further instances, the matching angles can, in some embodiments, assist in transitioning the guide head 1072 to the contracted state for proximal withdrawal of the guide member 1070 from the cannula 1041. In the illustrated embodiment, the distal tip 1043 of the cannula 1041 is recessed proximally from a distal tip of the catheter tube 104 when the access assembly 1030 is fully inserted into the catheter tube 104.

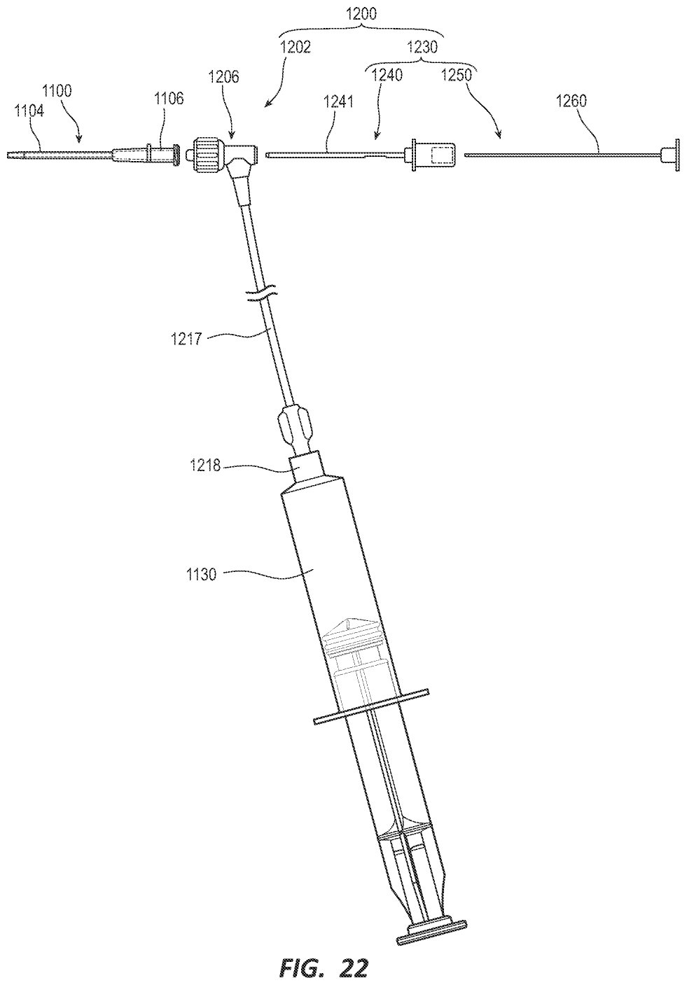

FIG. 22 depicts an embodiment of a base catheter system 1100 and an embodiment of an access system 1200 that can be used with the base catheter system 1100. The base catheter system 1100 can resemble the base catheter system 100 described previously, and the access system 1200 can resemble the access system 900 described previously, in many respects. In the illustrated embodiment, the base catheter system 1100 includes a catheter tube 1104 and a hub 1106. The access system 1200 includes an extension set 1202 and an access assembly 1230, which is shown in a disassembled state. The illustrated access assembly 1230 includes a cannula assembly 1240 that includes a cannula 1241 and an obturator assembly 1250 that includes a core or obturator 1260.

The core or obturator 1260 may function in manners similar to the core or obturator 960 discussed above. In the illustrated embodiment, a guide member (e.g., with an expandable/collapsible guide head) is omitted from the obturator assembly 1250. An outer diameter of the obturator 1260 may be larger, relative to an inner diameter of the cannula 1241, as compared with the outer diameter of the obturator 960 relative to the inner diameter of the cannula 941, due to the absence of a guide member. For example, in some embodiments, the obturator 1260 may fit closely or snugly within the cannula 1241 such that there is little or no space between the outer surface of the obturator 1260 and the inner surface of the cannula 1241. In some instances, a close fit may inhibit or prevent scratching, snagging, coring, tearing, or other damage to the catheter tube 1104 due to the catheter tube 1104 entering a space between the obturator 1260 and the cannula 1241. Stated otherwise, a close fit between the obturator 1260 and the cannula 1241 can facilitate movement of the access assembly 1230 through the catheter tube 1104 in a manner that provides little or no damage to the catheter tube 1104. The obturator 1260 may be said to fill the lumen of the cannula 1241 and/or may close the proximal opening 1248 of the cannula 1241.

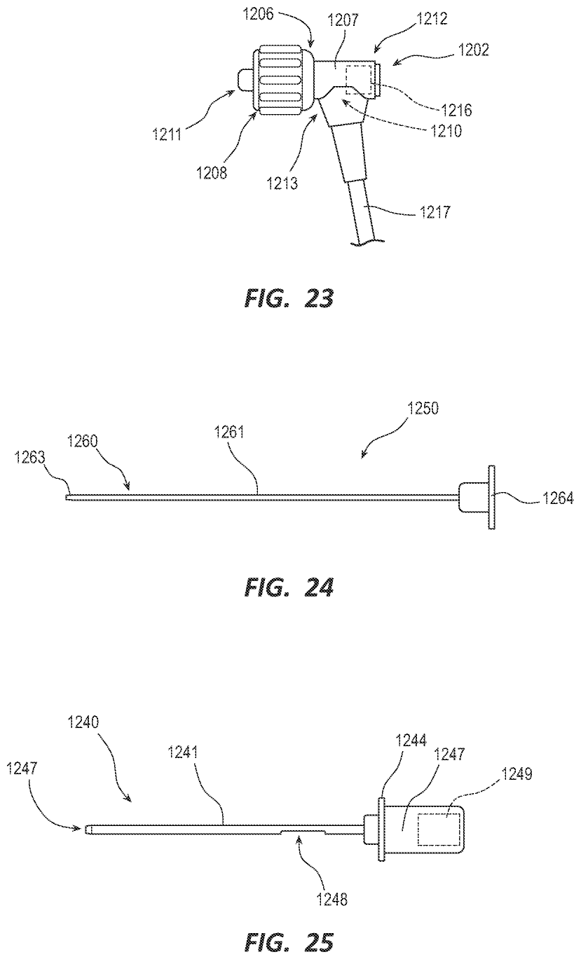

With reference to FIGS. 22 and 23, the extension set 1202 can include a hub 1206, an extension line 1217, and a connector 1218. The connector 1218 can be connected (e.g., selectively connected) with a fluid collection device 1130, such as a syringe or an evacuated tube. In other or further instances, the connector 1218 can be connected with a fluid delivery device for infusions, flushes, etc.

With reference to FIG. 23, in certain embodiments, the hub 1206 can include a housing 1207 that defines an inner chamber 1210. The hub 1206 can further include a first or distal port 1211, a second or proximal port 1212, and a third or side port 1213, each of which may be in fluid communication with the chamber 1210. The hub 1206 can include a connector 1208 for securing the hub 1206 to the base catheter system 1100. In the illustrated embodiment, the connector 1208 comprises a male luer lock connector. In the illustrated embodiment, the hub 1206 includes a sealing member 1216 associated with the proximal port 1212. The various components or features of the hub 1206 may function in manners such as described above with respect to like-named and like-numbered components or features of the hub 906.

With reference to FIG. 24, the obturator assembly 1250 can include the core or obturator 1260, as previously mentioned. The obturator 1260 can include an elongated rod 1261, which can include a tip 1263. The obturator 1260 can further include a proximal tab 1264. The various components or features of the obturator assembly 1250 may function in manners such as described above with respect to like-named and like-numbered components or features of the obturator assembly 960.

With reference to FIG. 25, the cannula assembly 1240 can include a cannula 1241. The cannula 1241 can include an elongated tube with a lumen therein that includes a distal port 1247 and a side port or proximal port 1248. The cannula assembly 1240 can further include a proximal handle or tab 1244. In the illustrated embodiment, the proximal tab 1244 includes an elongated proximal stem 1247, which may be suitable for gripping by a practitioner. The stem 1247 may include a sealing member 1249 therein, which may function in manners such as described above with respect to the sealing member 949. The various components or features of the cannula assembly 1240 may function in manners such as described above with respect to like-named and like-numbered components or features of the cannula assembly 940.

As with the cannula assembly 940, in some embodiments, the cannula assembly 1240 may be devoid of the sealing member 1249. For example, in some embodiments, the obturator assembly 1250 may not be used. The proximal handle 1244 may close off a proximal end of the cannula 1240 and prevent blood from egressing from the proximal end of the cannula 1240.

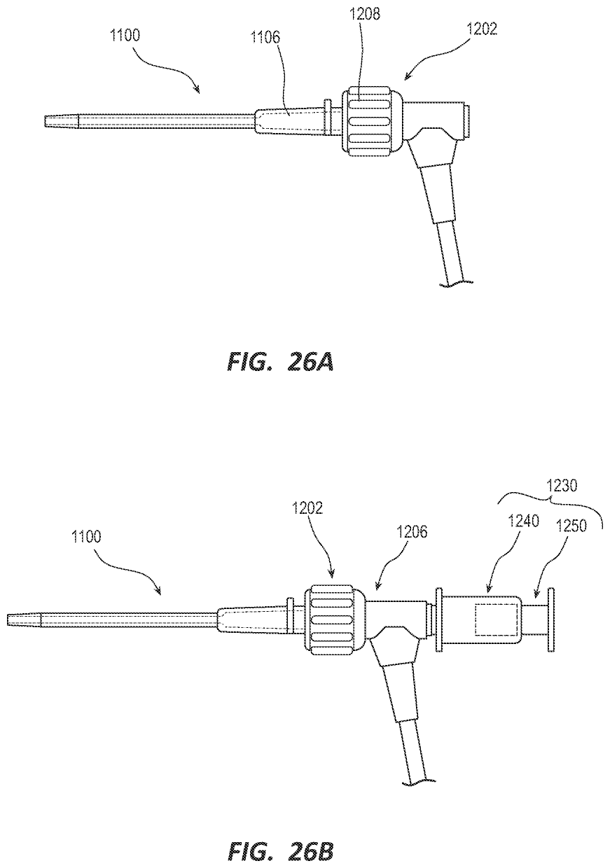

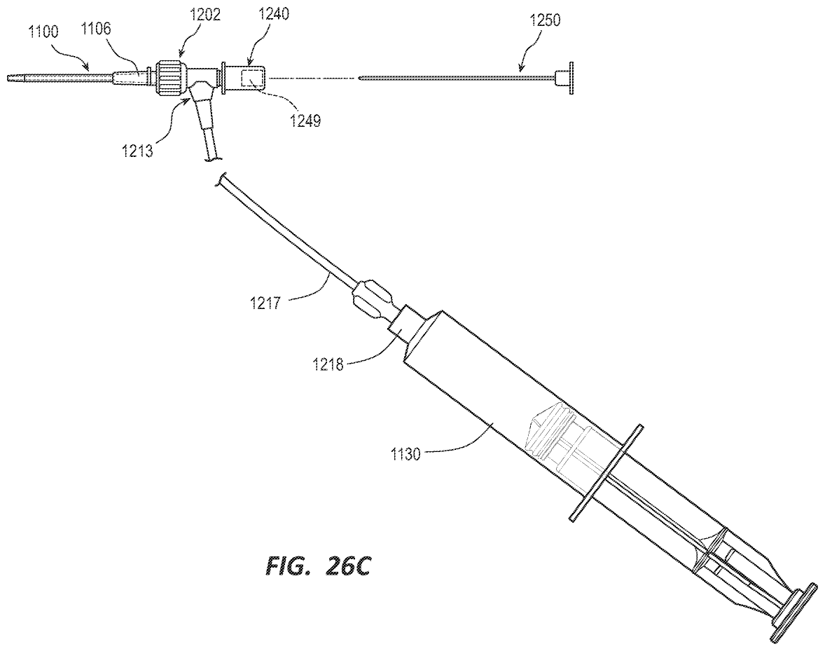

FIG. 26A depicts a stage of an illustrative method of using the access system 1230 with the base catheter system 1100. In the depicted stage, the extension set 1202 is securely attached to the hub 1106 of the base catheter system 1100 via the connector 1208.

FIG. 26B depicts another stage of the illustrative method in which the access assembly 1230 has been inserted through the hub 1206 of the extension set 1202 and into the base catheter system 1100. The obturator assembly 1250 and the cannula assembly 1240 can be coupled together prior to introduction of the access assembly 1230 into the base catheter system 1100.

FIG. 26C depicts another stage of the illustrative method in which the obturator assembly 1250 has been removed from the cannula assembly 1240, which remains coupled with the extension set 1202 and the base catheter system 1100. Upon removal of the obturator assembly 1250, the sealing member 1249 can naturally transition to a closed state to prevent blood from exiting through the proximal end of the cannula assembly 1240. Blood can be collected into the fluid collection device 1130. Blood can progress from the vessel, through the distal port 1247 of the cannula 1241 and through the lumen of the cannula 1241, through the proximal port 1248 of the cannula (see FIG. 25), through at least a portion of the chamber 1210 of the hub 1206 (see FIG. 23), through the side port 1213, through the extension line 1217, through the connector 1218, and into the fluid collection device 1130.

After a blood collection event, the cannula assembly 1240 can be removed from the extension set 1202. In some instances, the extension set 1202 may be maintained on the hub 1106. In some instances, a fluid flush may be performed via the extension set 1202, e.g., by connecting a fluid delivery device to the connector 1218 and flushing saline through the extension set 1202 and the base catheter system 1100. In some instances, the extension set 1202 may be removed from the hub 1106 after one, two, three, four, or more fluid collection events via the extension set 1202—e.g., in conjunction with one or more access assemblies 1230 individually inserted through the extension set 1202 and each operated in manners such as previously discussed—and in other or further instances, after one, two, three, four, or more fluid delivery events via the extension set 1202.

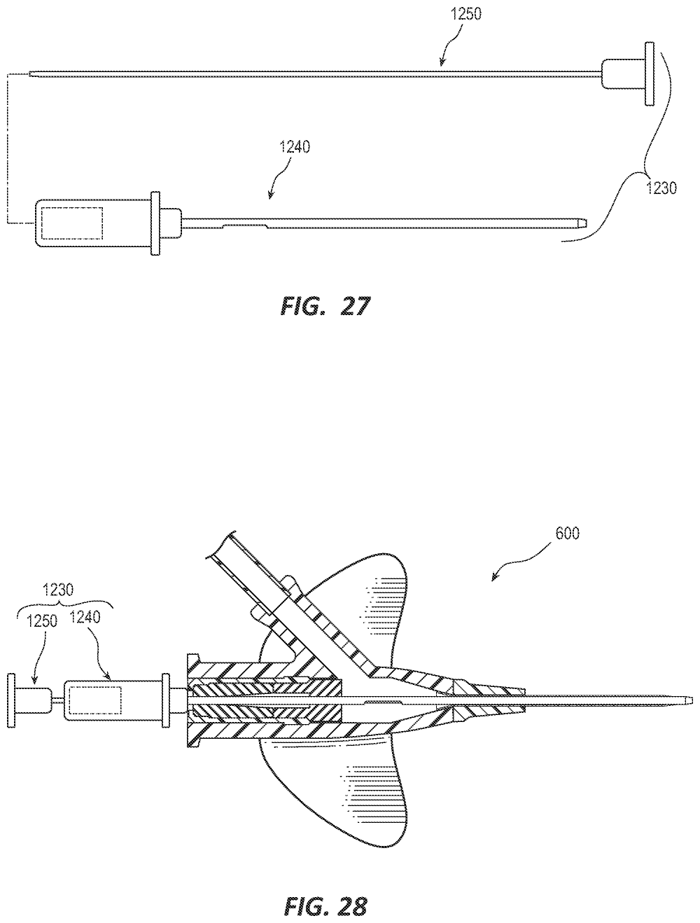

FIG. 27 is a perspective view of an embodiment of the access assembly 1230 that can be used with a closed base catheter system, such as an embodiment of the closed catheter system 600. In some embodiments, the access assembly 1230 may be used with each of an open catheter system, such as, for example, discussed above with respect to FIGS. 26A-26C, and a closed catheter system. The access assembly is shown in a disassembled state. The illustrated access assembly 1230 includes an embodiment of the cannula assembly 1240 and further includes an embodiment of the obturator assembly 1250 configured to be positioned within the cannula assembly 1240 and function in manners such as previously disclosed.

FIG. 28 is a cross-sectional view of the access assembly 1230 in an assembled state and positioned within the hub 706 of the closed catheter system 600 depicted in and described with respect to FIG. 18. Blood collection and/or infusions via the closed catheter system 600 can proceed in much the same manner as described above with respect to the base catheter 1100 with an extension set 1202 attached thereto. As previously discussed, the illustrated closed catheter system 600 may be viewed as having an extension set integrally incorporated therein. After a blood collection event, the cannula assembly 1240 can be removed.

The present paragraph recites 32 illustrative examples of systems that correspond to the foregoing written description and/or the illustrative drawings. In these examples, the terminology “Example X to Example Y” means Example X through Example Y, and thus includes the endpoints of the recited range of examples.
Example 1. An Access System Comprising:
   a cannula configured to be inserted into a catheter system that comprises a hub comprising an internal chamber, a distal port in fluid communication with the internal chamber and coupled with a catheter tube that is configured to be preplaced in a blood vessel of a patient, and an access port in fluid communication with the internal chamber,
   the cannula comprising a lumen, a distal opening at a distal end of the cannula that is in fluid communication with the lumen, and a proximal opening that extends through a sidewall of the cannula and is in fluid communication with the lumen,
   the cannula being insertable through at least a portion of the catheter tube to a collection orientation in which, when the catheter tube is within the blood vessel of the patient:
      the distal opening of the cannula is positioned within the blood vessel of the patient and is in fluid communication with an interior of the blood vessel to permit blood to flow into the lumen of the cannula; and
      the proximal opening of the cannula is positioned within the internal chamber of the hub of the catheter system to permit blood to flow from the lumen of the cannula into the internal chamber of the hub and through the access port of the hub.
Example 2. The access system of Example 1, further comprising a handle coupled to a proximal end of the cannula, wherein each of the handle and the proximal opening of the cannula are configured to remain at an exterior of the patient when the cannula is inserted into the catheter tube when preplaced into the vessel.
Example 3. The access system of Example 2, wherein the cannula comprises a utility port at a proximal end thereof, and wherein the handle comprises a sealing member configured to selectively close the utility port of the cannula.
Example 4. The access system of Example 3, further comprising an obturator that extends through the sealing member of the handle and through the utility port and the distal port of the cannula.
Example 5. The access system of Example 4, wherein the obturator is selectively removable from the cannula and the handle, and wherein sealing member automatically closes the utility port of the cannula upon removal of the obturator.
Example 6. The access system of any one of Examples 1 to 3, further comprising an obturator that extends through the cannula.
Example 7. The access system of Example 6, wherein the obturator is more compliant than the cannula.
Example 8. The access system of Example 6 or Example 7, wherein the obturator fills the lumen of the cannula and closes the proximal opening of the cannula when the obturator extends through the cannula.
Example 9. The access system of Example 1, further comprising an obturator configured to extend through the cannula such that a distal tip of the obturator extends distally past a distal tip of the cannula, the obturator being selectively removable from the cannula.
Example 10. The access system of Example 9 further comprising a spacer positioned between the obturator and the cannula, the spacer member being selectively removable from the cannula.
Example 11. The access system of Example 10, wherein a distal end of the spacer is selectively expandable and collapsible.
Example 12. The access system of Example 11, wherein the distal end of the spacer is configured to expand upon insertion of the obturator therethrough.
Example 13. The access system of Example 10 or Example 11, wherein the distal end of the spacer is configured to collapse upon removal of the obturator therefrom.
Example 14. The access system of any one of Examples 1 to 13, wherein the hub of the catheter system comprises a proximal port in fluid communication with the internal chamber.
Example 15. The access system of Example 14, wherein the proximal port of the hub comprises a sealing member that is configured to provide a fluid-tight seal against the cannula when the cannula extends therethrough.
Example 16. The access system of Example 15, wherein the sealing member is configured to self-seal upon removal of the cannula therefrom.
Example 17. The access system of any one of Examples 1 to 16, further comprising the catheter system.
Example 18. The access system of Example 17, wherein the catheter system is an open catheter system.
Example 19. The access system of Example 18, wherein the catheter system comprises an additional hub configured to couple with the distal port, wherein the additional hub is attached to the catheter tube.
Example 20. The access system of Example 17 or Example 18, wherein an extension set that is selectively couplable to and decouplable from the catheter tube comprises the hub.
Example 21. The access system of Example 17, wherein the catheter system is a closed intravenous catheter system.
Example 22. The access system of any one of Examples 1 to 21, further comprising a guide configured to couple with the catheter system and constrain movement of the cannula as the catheter is advanced distally toward and then through the catheter tube.
Example 23. The access system of any one of Examples 1 to 22, further comprising a valve assembly configured to prevent blood from flowing through the proximal opening of the cannula when in a closed state and configured to permit blood to flow through the proximal opening of the cannula when in an open state.
Example 24. The access system of Example 23, wherein the valve assembly is further configured to prevent blood from flowing through the lumen of the cannula when the valve assembly is in the closed state.
Example 25. The access system of Example 23, wherein the valve assembly comprises an obturator that extends through the lumen.
Example 26. The access system of Example 23, wherein the valve assembly is configured to rotate to transition between the closed and open states.
Example 27. The access system of Example 23, wherein the valve assembly is configured to translate relative to a longitudinal axis of the cannula to transition between the closed and open states.
Example 28. The access system of Example 27, further comprising a handle attached to the cannula and coupled to the valve assembly, the handle being configured to prevent the valve assembly from decoupling from the handle when the valve assembly transitions to the open state.
Example 29. A blood collection access system comprising:
   a cannula sized to be inserted through at least a portion of a catheter tube of a catheter system that is preplaced into a vessel of a patient, the cannula comprising a lumen, a distal opening in fluid communication with the lumen at a distal end of the cannula, and a proximal opening through a sidewall of the cannula that is in fluid communication with the lumen and is positioned so as to remain outside of the patient when the cannula is inserted into the catheter tube when preplaced into the vessel.
Example 30. The blood collection access system of Example 29, further comprising an obturator configured to extend through the cannula such that a distal tip of the obturator extends distally past a distal tip of the cannula, the obturator being selectively removable from the cannula.
Example 31. The blood collection access system of Example 30, further comprising a spacer positioned between the obturator and the cannula, the spacer member being selectively removable from the cannula.
Example 32. The blood collection access system of Example 29, further comprising an extension set.

The term “coupled to” can mean connected to in any suitable fashion, whether that coupling is direct or indirect. Separate components may be coupled to each other. Moreover, in some instances, where separately identified components are integrally formed from a unitary piece of material, or stated otherwise, are included together in a monolithic element, those elements may also be said to be coupled to one another.

Although the foregoing detailed description contains many specifics for the purpose of illustration, a person of ordinary skill in the art will appreciate that many variations and alterations to the following details can be made and are considered to be included herein. Accordingly, the foregoing embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description. These additional embodiments are determined by replacing the dependency of a given dependent claim with the phrase “any of claims [x] through the immediately preceding claim,” where the bracketed term “[x]” is replaced with the number of the most recently recited independent claim. For example, for the first claim set that begins with independent claim 1, claim 3 can depend from either of claims 1 and 2, with these separate dependencies yielding two distinct embodiments; claim 4 can depend from any one of claim 1, 2, or 3, with these separate dependencies yielding three distinct embodiments; claim 5 can depend from any one of claim 1, 2, 3, or 4, with these separate dependencies yielding four distinct embodiments; and so on.

Recitation in the claims of the term “first” with respect to a feature or element does not necessarily imply the existence of a second or additional such feature or element. Elements specifically recited in means-plus-function format, if any, are intended to be construed in accordance with 35 U.S.C. § 112(f). Elements not presented in requisite means-plus-function format are not intended to be construed in accordance with 35 U.S.C. § 112(f). Embodiments of the invention in which an exclusive property or privilege is claimed are defined as follows.

1. An access system comprising:
a cannula configured to be inserted into a catheter system that comprises:
a hub comprising an internal chamber;
a distal port in fluid communication with the internal chamber and coupled with a catheter tube that is configured to be preplaced in a blood vessel of a patient; and
an access port in fluid communication with the internal chamber,
the cannula comprising a lumen, a distal opening at a distal end of the cannula that is in fluid communication with the lumen, and a proximal opening that extends through a sidewall of the cannula and is in fluid communication with the lumen,
the cannula being insertable through at least a portion of the catheter tube to a collection orientation in which, when the catheter tube is within the blood vessel of the patient:
the distal opening of the cannula is positioned within the blood vessel of the patient and is in fluid communication with an interior of the blood vessel to permit blood to flow into the lumen of the cannula; and
the proximal opening of the cannula is positioned within the internal chamber of the hub of the catheter system to permit blood to flow from the lumen of the cannula into the internal chamber of the hub and through the access port of the hub.

2. The access system of claim 1, further comprising a handle coupled to a proximal end of the cannula, wherein each of the handle and the proximal opening of the cannula are configured to remain at an exterior of the patient when the cannula is inserted into the catheter tube when preplaced into the vessel.

3. The access system of claim 2, wherein the cannula comprises a utility port at a proximal end thereof, and wherein the handle comprises a sealing member configured to selectively close the utility port of the cannula.

4. The access system of claim 3, further comprising an obturator that extends through the sealing member of the handle and through the utility port and the distal port of the cannula.

5. The access system of claim 4, wherein the obturator is selectively removable from the cannula and the handle, and wherein sealing member automatically closes the utility port of the cannula upon removal of the obturator.

6. The access system of claim 1, further comprising an obturator that extends through the cannula.

7. The access system of claim 6, wherein the obturator fills the lumen of the cannula and closes the proximal opening of the cannula when the obturator extends through the cannula.

8. The access system of claim 1, further comprising an obturator configured to extend through the cannula such that a distal tip of the obturator extends distally past a distal tip of the cannula, the obturator being selectively removable from the cannula.

9. The access system of claim 8 further comprising a spacer positioned between the obturator and the cannula, the spacer being selectively removable from the cannula.

10. The access system of claim 9, wherein the distal end of the spacer is configured to collapse upon removal of the obturator therefrom.

11. The access system of claim 1, wherein the hub of the catheter system comprises a proximal port in fluid communication with the internal chamber.

12. The access system of claim 11, wherein the proximal port of the hub comprises a sealing member that is configured to provide a fluid-tight seal against the cannula when the cannula extends therethrough.

13. The access system of claim 12, wherein the sealing member is configured to self-seal upon removal of the cannula therefrom.

14. The access system of claim 1, further comprising the catheter system.

15. The access system of claim 14, wherein the catheter system is an open catheter system.

16. The access system of claim 15, wherein the catheter system comprises an additional hub configured to couple with the distal port, wherein the additional hub is attached to the catheter tube.

17. The access system of claim 14, wherein an extension set that is selectively couplable to and decouplable from the catheter tube comprises the hub.

18. The access system of claim 14, wherein the catheter system is a closed intravenous catheter system.

19. The access system of claim 1, further comprising a valve assembly configured to prevent blood from flowing through the proximal opening of the cannula when in a closed state and configured to permit blood to flow through the proximal opening of the cannula when in an open state.

20. The access system of claim 19, wherein the valve assembly is further configured to prevent blood from flowing through the lumen of the cannula when the valve assembly is in the closed state.

21. The access system of claim 19, wherein the valve assembly comprises an obturator that extends through the lumen.

22. The access system of claim 19, wherein the valve assembly is configured to rotate to transition between the closed and open states.

23. The access system of claim 19, wherein the valve assembly is configured to translate relative to a longitudinal axis of the cannula to transition between the closed and open states.

24. The access system of claim 23, further comprising a handle attached to the cannula and coupled to the valve assembly, the handle being configured to prevent the valve assembly from decoupling from the handle when the valve assembly transitions to the open state.
